(19) United States
(12) Patent Application Publication
(10) Pub. No.: US 2020/0362052 A1
(43) Pub. Date: Nov. 19, 2020

(54) COMPOSITIONS AND METHODS FOR TREATING TOLL-LIKE RECEPTOR-DRIVEN INFLAMMATORY DISEASES

(71) Applicant: Souvie Biodelivery, LLC, Tucson, AZ (US)

(72) Inventor: Souvenir Tachado, Tucson, AZ (US)

(21) Appl. No.: 16/986,063

(22) Filed: Aug. 5, 2020

Related U.S. Application Data
(63) Continuation-in-part of application No. PCT/US19/25207, filed on Apr. 1, 2019.
(60) Provisional application No. 62/882,963, filed on Aug. 5, 2019.
(60) Provisional application No. 62/651,621, filed on Apr. 2, 2018.

(51) Int. Cl. C07K 16/28; C07K 14/705; A61P 29/00

(52) U.S. Cl. CPC C07K 16/2896; C07K 14/70596; A61K 38/00; C07K 2319/30; A61P 29/00

(57) ABSTRACT

Many disorders wherein inflammation is a hallmark such as rheumatoid arthritis, sepsis, or cancer, are chronic and impose a significant burden on family and society due to their high morbidity and mortality. Each year the United States government spends $2.6T to treat chronic Inflammatory diseases, which are linked to 70% of the deaths every year. Protein therapeutics, such as anti-TNFα antibodies that selectively block the TNFα cascade, have become the mainstay therapy for the management of chronic inflammatory diseases. Despite the promise of these early studies, concerns about the side effects of these protein drugs, including induction of auto-antibodies and immuno-suppression, may limit the applications to disease treatments. Thus, there is a need to develop new drugs and delivery systems for the prevention and treatment of inflammatory diseases. The present invention relates to extracellular vesicle composition, system, and methods for treating Toll-like receptor-driven inflammatory disease.

### CROSS-REFERENCES TO RELATED APPLICATIONS

This application is a non-provisional and claims benefit of U.S. Provisional Application No. 62/882,963 filed Aug. 5, 2019, the specification(s) of which is/are Incorporated herein in their entirety by reference.

This application is a continuation-in-part of PCT/US2019/025207 filed Apr. 1, 2019, which claims the benefit of the U.S. Provisional Application No. 62/651,621 filed Apr. 2, 2018, the specification(s) of which is/are incorporated herein in their entirety by reference.

### REFERENCE TO A SEQUENCE LISTING

Applicant asserts that the paper copy of the Sequence Listing is identical to the Sequence Listing in computer readable form found on the accompanying computer file, entitled SOUV_19_01_NP_Sequencing_Listing_ST25. The content of the sequence listing is incorporated herein by reference in its entirety.

### FIELD OF THE INVENTION

The present invention relates to extracellular vesicle composition, system and methods for treating Toll-like receptor-driven inflammatory disease.

### BACKGROUND OF THE INVENTION

Inflammation is a hallmark of many debilitating and life-threatening disorders, including inflammatory disease, rheumatoid arthritis, psoriasis, sepsis, atherosclerosis, Alzheimer's disease, and cancer. Many of these disorders are chronic and impose a significant burden to family and society due to their high morbidity and mortality. Each year the United States government spends $2.6T to treat chronic inflammatory diseases, which are linked to 70% of the deaths every year. Endosomal Toll-like receptors (TLR3, TLR7, TLR8 and TLR9), which are activated by molecules originating from either endogenous or exogenous sources, are important mediators of inflammation. Endogenous sources of inflammation include the release of mitochondrial DNA, which activates TLR9. Exogenous sources include viral RNA or microRNAs, which activate TLR3 or TLR7/8, respectively. Cell surface expressed TLRs include TLR1, TLR2, TLR4, TLR5, TLR6 and TLR10. They recognize bacterial lipoproteins (TLR 2/6), flagellin (TLR5), lipopolysaccharides (TLR4). Preventing these TLR-mediated inflammatory responses could reduce the health care burdens in the United States.

While many genetic and environmental factors play a prominent role in disease progression, the proinflammatory cytokines such as TNFα, IL-1@, IL-6. IL-12, IL-18, IL-21, IL-22. IL-26, IFN-© are recognized as the central players in eliciting proinflammatory cascades. Protein therapeutics, such as anti-TNFα antibodies that selectively block the TNFα cascade, has become the mainstay therapy for the management of chronic inflammatory diseases.

Despite the promise of the early studies, concerns about the side effects of these protein drugs, including induction of auto-antibodies and immuno-suppression, may limit the applications to disease treatments.

Furthermore, the efficacy of antibody-based therapies is limited by their rapid clearance and poor penetration of tissues. Thus, there is a need to develop new drugs and delivery systems for the prevention and treatment of inflammatory diseases.

### BRIEF SUMMARY OF THE INVENTION

It is an objective of the present invention to provide an engineered extracellular vesicle composition as well as a method of use that allows for the treatment of Toll-like receptor driven inflammatory diseases, as specified in the independent claims. Embodiments of the invention are given in the dependent claims. Embodiments of the present invention can be freely combined with each other if they are not mutually exclusive.

The present invention may feature an engineered extracellular vesicle, preferably exosomes, comprising a fusion protein having tetraspanin, or functional fragment thereof, linked to anti-TLR antibody, or functional fragment thereof, that serve as a targeting moiety and inhibits the activation of a cell surface expressed TLR to induce proinflammatory responses. In some embodiments the anti-TLR antibody is expressed on the exterior of the engineered extracellular vesicle.

The present invention may feature a pharmaceutical composition comprising an engineered extracellular vesicle. In some embodiments, the engineered extracellular vesicle comprises a fusion protein having tetraspanin, or a functional fragment thereof, linked to an anti-TLR antibody, or a functional fragment thereof, that inhibits a cell surface expressed TLR, and a pharmaceutically acceptable carrier or excipient.

Additionally, the present invention may feature a method of preventing the production of anti-drug antibodies, in an individual treated with biologics or a method of preventing immunosuppression in an individual treated with anti-TNFα biologic.

The present Invention may feature an exosome-based delivery system for delivering a therapeutic molecule to a cell. In some embodiments, delivery system comprises a fusion protein comprising a therapeutic molecule comprising an anti-TLR antibody, or functional fragment thereof, fused with a tetraspanin, or functional fragment thereof. In some embodiments, the tetraspanin, or functional fragment thereof, is positioned at a surface of an exosome. In some embodiments, the anti-TLR antibody, or functional fragment thereof will serve as a targeting moiety and neutralizing its effects by inhibiting dimerization of TLR receptors thereby dampening proinflammatory responses. The anti-TLR antibody serves a dual function: as a targeting moiety and inhibition of signaling cascade driven by TLR activation. The end result is dampening of proinflammatory responses.

Furthermore, the present invention may feature a dominant-negative engineered extracellular vesicle, wherein said extracellular vesicle comprises: a tetraspanin, or functional fragment thereof; a cleavable linker; and a dominant-negative fragment of a TLR receptor

There is a substantial need for an alternative drug treatment for chronic inflammatory autoimmune diseases. TNFα inhibitors remain the gold standard of biological therapies for autoimmune diseases such as rheumatoid arthritis (RA), even though approximately 30% of patients show no significant improvement with TNFα inhibitors. In addition, some patients become refractory to treatment over time, due to the production of inhibitory antibodies against anti-TNFα biologicals as well as immunosuppression. This maladaptive response may negate their therapeutic effectiveness in chronic disease conditions, which require prolonged drug treatment. Described herein are engineered extracellular vesicles, e.g., engineered exosomes, and methods of use, which avoid the production of anti-drug antibodies (ADAs), because the anti-endosomal TLR antibody (payload) is protected inside the exosomes and treated as “self” by the immune system, avoiding rapid clearance and reducing toxicity associated with the current drug delivery systems (DDS).

Despite showing promising applications, many of these synthetic DDS face issues of poor biocompatibility, sub-optimal targeting specificity and poor tissue penetration. In contrast, exosomes are endosomally-derived nanoparticles that are secreted by a variety of cell types and tissues and can specifically transfer material to recipient cells in a targeted manner. The exosome is one of nature's ways of delivering different proteins to desired cell-recipients, thereby providing the advantage of biocompatibility and reduced clearance rates. Provided herein are engineered exosomes, systems, and methods for targeting anti-inflammatory agents to interact solely with inflammatory cells, thereby avoiding unwanted off-target side-effects that can affect the efficacy or toxicology profile (FIG. 1).

An important requirement for endosomal TLR activation is cleavage of the TLR ectodomains by specific proteases, such as cathepsins, via their Z-loops when they reach the endosomal compartment from the endoplasmic reticulum. The methods, engineered extracellular vesicles, and systems provided herein block TLR ligand interaction by delivering exosomal-encapsulated anti-TLR antibodies (neutralizing antibodies) to endosomal compartments.

Proteolytic cleavage by endosome-resident proteases (cathepsins, AEP, furin-like proteases, and the like; Table 1) will then release these antibody-based TLR antagonists from their exosomal bound sites to inhibit TLR receptor dimerization, activation and induction of proinflammatory responses.

One of the unique and inventive technical features of the present invention is positioning the anti-TLR antibody on the outside surface of the engineered extracellular vesicle. Without wishing to limit the invention to any theory or mechanism, it is believed that the technical feature of the present invention advantageously provides the engineered extracellular vesicle not only the ability to hone in on specific target cells expressing cell surface TLR receptors, but also allows the engineered extracellular vesicle bearing the neutralizing antibody the ability to directly neutralize the target diseased cells. The advantage of such method is specific cell targeting thereby eliminating side effects. Additionally, the use of an engineered exosome allows for an increase in biocompatibility as exosomes are secreted by various cell types and tissues and specifically transfer material to recipient cells in a targeted manner. None of the presently known prior references or work has the unique inventive technical feature of the present invention.

Furthermore, the prior references teach away from the present Invention. For example, as previously discussed, constraints affecting nanotechnologies delivering of molecular cargo to specific cells or tissues include: 1) biocompatibility 2) molecular cargo loading efficiency, 3) specificity of the delivery platform to cells or tissue of interest. Furthermore, the inventive technical features of the present invention contributed to a surprising result. For example, instead of neutralizing the effects of TLR-triggered induction of proinflammatory responses, it enhances it leading to cytokine storms.

Additionally, the present invention may also provide a unique advantages by the methods and compositions described herein include that: the payload is protected from degradation by encapsulation in exosomal vesicles in the case of endosomal TLRs and has the potential of avoiding the formation of ADAs; specific targeting to the intended inflammatory cells (while avoiding off-target cells) is achieved using monocyte-derived exosomes, which display the immunologically-appropriate repertoire of surface antigens; the payload is taken up and trafficked to the correct intracellular compartment (endosomes), thus avoiding off-target effects in other sub-cellular compartments: the unique and inventive features of the present invention is the linker peptide is cleaved by intra-endosomal proteases/endopeptidases, or the like, to yield free payload in the endosomal compartment; the payload specifically binds an intracellular target receptor (e.g., a toll-like receptor (“TLR”)) and blocks pro-inflammatory signaling; the treatment is suitable for acute inflammatory indications, since the payload can remain temporarily active (1-2 hours) until endosomal processes degrade the payload, thus bringing to an end the anti-inflammatory effect of the composition; and the treatment is suitable for chronic inflammatory indications with repeated dosing.

Any feature or combination of features described herein are included within the scope of the present Invention provided that the features included in any such combination are not mutually inconsistent as will be apparent from the context, this specification, and the knowledge of one of ordinary skill in the art. Additional advantages and aspects of the present invention are apparent in the following detailed description and claims.

### INCORPORATION BY REFERENCE

Herein, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic of surface engineering of exosome via tetraspanin proteins. Peptides can be fused with CD63 either at the amino or carboxy terminus or on Inner loops or outer loops of CD63.

FIG. 1B shows the delivery of CD63 fusion genes into living cells will partition the displayed the engineered exosomes inside the exosome via CD63 anchoring.

FIGS. 1C-D show how engineered exosomes are released into the culture medium that can be recovered with polymer-based precipitation solution and centrifugation.

FIG. 2A shows mammalian expression vector and configuration of CD63-linker-anti-TLR antibody. Promoter sequences derived from cytomegalovirus (CMV) or elongation factor alpha (EF1.) are fused to drive expression of the fusion and puromycin-resistance genes respectively.

FIG. 2B shows a schematic illustration of vectors expressing chimera containing anti-TLR7 antibody or a control containing RFP or GFP.

FIG. 3 shows TLR signaling assay for rapid screening of antibodies. Transferrin-mediated uptake of protein complexes to the endosome delivers antibodies that are released upon acidification. Free antibodies bind TLR receptor and block signaling in reporter cells. Inhibition of signaling decreases the secretion of alkaline phosphatase enzyme (SEAP) into culture medium. SEAP enzymatic activity will be measured using substrate 4-methylumbelliferyl phosphate (4-MUP) to detect fluorescent 4-methylumbelliferone (4-MU) via microplate spectrofluorimeter. SA, streptavidin or NeutrAvidin. Btn, biotin.

FIGS. 4A-4C show protein complexes bearing anti-TLR9 antibody inhibit TLR9 signaling in receptor cells. FIG. 4A shows HEK-TLR9 NFkB cells treated with CpG DNA oligo (ODN 2006) in LyoVec secrete SEAP into the medium (as shown by the initial rate of 4-MUP hydrolysis by AP enzyme), whereas HEK-NFkB control cells do not. FIG. 4B shows HEK-TLR9 NFkB cells treated with CpG (10 μg/mL In LyoVec) secrete SEAP into the medium, but pre-treatment with protein complexes (containing endosome-trafficked transferrin) that bear the anti-TLR9 polyclonal antibody payload inhibited SEAP secretion in a concentration-dependent manner. Protein complexes bearing non-specific IgG had no effect. FIG. 4C shows neither the TLR7 agonist imiquimod nor LyoVec could stimulate TLR9 signaling in HEK-TLR9 NFkB cells. Pre-treatment with free antibody (either non-specific IgG or ant-TLR9 antibody) did not Inhibit CpG/LyoVec-stimulated TLR9 signaling. Pre-treatment with the protein complex armed with control IgG did not inhibit TLR9 signaling with CpG/LyoVec, but by comparison, protein complexes loaded with anti-TLR9 antibody payload demonstrated inhibition of signaling in a concentration dependent manner with 9 nM and 18 nM anti-TLR9 complex (* p<0.05; * p<0.01).

FIG. 5 shows anti-TLR antibody (Ab) inhibits imiquimod-induced NFkB activation in a dose dependent manner in reporter cells. Western blot analysis of reporter cells following Inhibition of phospho IκB with anti-TLR9 Ab and incubated with imiquimod. Membranes were probed with anti-phospho IκB and immunoreactive bands were detected with HRP-conjugated secondary antibody. Total IκB was used to validate protein loading equivalence after stripping the membrane. Western blot is a representative experiment of one or four independent experiments with similar results.

FIG. 6 shows the centrifugation-precipitation protocol for exosome isolation.

FIG. 7 shows engineered exosomes bearing anti-TLR4 Ab targets TLR4 receptor, that inhibits LPS-driven inflammatory response.

### DETAILED DESCRIPTION OF THE INVENTION

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a,” “an,” and “the” include plural referents unless the context clearly indicates otherwise. For example, reference to “a protein” includes a plurality of proteins; reference to “a cell” includes populations of a plurality of cells.

Extracellular Vesicles:

Provided herein, are engineered extracellular vesicles, preferably an engineered exosome, comprising a fusion protein having tetraspanin, or a functional fragment thereof, linked to an anti-TLR antibody, or a functional fragment thereof, wherein the anti-TLR antibody inhibits an endosomal TLR activity. In some embodiments, the extracellular vesicle is selected from an exosome, ectosome or microvesicle. In a particular embodiment, the extracellular vesicle is an exosome.

The terms “extracellular vesicle” or “EV” as used herein shall be understood to relate to any type of vesicle that is obtainable from a cell in any form, including for example, a microvesicle (e.g. any vesicle shed from the plasma membrane of a cell), an exosome (by exocytosis of multivesicular bodies [MVBs], ectosomes (by shedding of the cell surface membrane), apopttic bodies, nanoparticles, microparticles, shedding vesicles, shedding bodies, exovesicles, and the like. As used herein, the phrase “engineered extracellular vesicle” refers to an EV, or vesicle, that includes at least one or more proteins that are not naturally occurring in an exosome, or that do not naturally occur together in an extracellular vesicle, such an exosome. In some embodiments, the extracellular vesicle used herein is an Exosome.

As used herein, the phrase “engineered exosome” refers to an exosome, or vesicle, that Includes at least one or more proteins that are not naturally occurring in an exosome, or that do not naturally occur together in an exosome. Exosomes are natural cell-derived extracellular vesicles with a diameter of about 40-120 nm that originate from internal endocytic compartments, multivesicular bodies, and participate in intercellular communication as well as transport of genetic material. Because of their ideal native structure and characteristics, exosomes are contemplated herein to be promising nanocarriers for clinical use. Compared to synthetic nanoformulations, exosomes provide the advantage of enhancing delivery and therapeutic efficacy due to their native biocompatibility in vivo. Exosomes are lipid bilayer vesicles that form intracellularly upon inward invagination of endosome membranes and are generated as intraluminal vesicles by reverse budding of membrane multivesicular bodies (MVBs). Fusion of MVBs limiting membrane with the plasma membrane triggers the release of exosomes into the extracellular milieu. Exosomes incorporate messenger RNAs (mRNAs), micro RNAs (miRNAs), proteins, and lipids, that are functional in target cells, miRNAs transferred by tumor-derived exosomes downregulate the TAK signaling pathway in hepatocarcinogenesis and are pivotal in promoting tumor metastasis via proinflammatory cytokine-driven expansion of myeloid-derived suppressor cells. Exosomal Epstein Barr virus (EBV) miRNAs induce a concentration-dependent repression of an immunoregulatory gene CXCL11/ITAC, inducing EBV-associated lymphomas. Exosomes isolated from HIV-infected cells and sera from HIV-infected persons were shown to contain HIV-TAR miRNA and regulate apoptosis in bystander cells. It has been demonstrated that HIV produces novel miRNAs associated with exosomes derived from HIV-infected macrophages and sera from HIV-infected persons (see Bernard et al., Novel HIV-1 miRNAs stimulate TNF(release in human macrophages via TLR8 signaling pathway. PLoS One. 2014; 9(9):e106006. PubMed PMID: 25191859: PubMed Central PMCID: PMC4156304).

Exosomes are engineered at the cellular level under natural conditions. In accordance with the present invention, exosomes are provided having and anti-TLR cargo or payload, wherein the engineered exosome serves as a cell-derived gene delivery vehicle carrying the anti-TLR therapeutic proteins (biologics) for treating various diseases caused by endosomal or surface receptor TIR-driven inflammation, as well as for treating cytokine storm.

As used herein, the term “microvesicle” (also referred to as, circulating microvesicles, or microparticles) or refers to a type of extracellular vesicle (EV), between about 25 to about 1500 nanometers (nm) in diameter, or in other embodiments between about 50 and 1,000 nm in diameter, found in many types of body fluids as well as the interstitial space between cells. Microvesiles are circular fragments of plasma membrane ranging from 100 nm to 1000 nm shed from almost all cell types. Microvesicles play a role in intercellular communication and can transport mRNA, miRNA, lipids, and proteins between cells. Microvesicles have been implicated in the process of a remarkable anti-tumor reversal effect in cancer, tumor Immune suppression, metastasis, tumor-stroma interactions and angiogenesis along with having a primary role in tissue regeneration. They originate directly from the plasma membrane of the cell and reflect the antigenic content of the cells from which they originate. Microvesicles have a role in cell signaling and the process of molecular communication between cells, and are released by a number of cell types. Microvesiles are generally considered to be a heterogeneous population of exosomes (<100 nm) and shed microvesicles (100-1000 nm), which are similar but have distinct mechanisms of formation. Through these mechanisms, microvesicles are released into the extracellular space and interact with specific target cells, delivering bioactive molecules.

As used herein, the term “ectosorme” refers to a type of extracellular vesicle (EV) that is assembled in the plasma membrane; and are discrete plasma membrane domains marked by layers of dense material associated to their cytosolic surface. Ectosome domains undergo outward budding followed by their pinching off as round vesicles.

As used herein, the term “tetraspanin,” or “tetraspnnin,” or “TSPAN,” used herein interchangeably, refers to a protein belonging to the transmembrane 4 superfamily (TM4SF) of proteins. Tetraspanins possess four transmembrane domains, intracellular N- and C-termini, and two extracellular domains: one short extracellular domain, called the “small extracellular domain or loop,” “SED/SEL” or “EC1”; and one longer extracellular domain (typically, about 100 amino acid residues in length), called the “large extracellular domain/loop,” “LED/LEL,” or “EC2.” Typically, there are four or more cysteine residues in the EC2 domain, with two in a highly conserved “CCG” motif. Tetraspanins are characterized by conserved domains listed in the Conserved Domains database of the National Center for Biotechnology Information (NCBI) under pfam00335. Tetraspanins are involved in diverse physiological processes, such as cell activation and proliferation, adhesion and motility, differentiation, oncogenesis, and others. A “functional fragment” of a tetraspanin is a subpart of a tetraspanin molecule that still maintains at least one biological function of the complete tetraspanin, for example, binding a natural ligand of the tetraspanin. Examples of tetraspanins (or functional fragments thereof) useful in practicing the invention include, but are not limited to: TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 1B), TSPAN21 (uroplakin1A) TSPAN22 (peripherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, and TSPAN34. In a particular embodiment, the tetraspanin is TSPAN30 (CD63).

In some embodiments, the fusion protein for use herein is a tetraspanin-anti-TIR antibody fusion protein, wherein the anti-TLR moiety, or fragment thereof, is fused within one of the extracellular loops (e.g. EC2 domain, NH2 domain) of the tetraspanin protein.

The term “endosomal TLR” refers to an intracellular toll-like receptor in an endosome. Nucleic-acid sensing TIRs reside in the endosome and consist of TLR3 (recognizing viral dsRNA); TLR7 and TLR8 (recognizing viral ssRNA): and TLR9 (recognizing bacterial and viral unmethylated CpG-containing DNA motifs). The restricted access to these intracellular TLRs is one mechanism that prevents them from being aberrantly activated by self-nucleic acids under physiological conditions. However, nucleic acids released from apoptotic cells can activate these TLRs and instigate autoimmune diseases. Moreover, nucleic acids derived from viruses during cytosolic replication can be transported into endosomes during the process of autophagy where they activate endosomal TLRs. Under normal physiological conditions, the localization of endosomal TLRs to intracellular compartments prevents their activation by self-nucleic acids, because self-nucleic acids from dead cells in damaged tissues are unable to passively permeate across other cells and enter into endosomes. In contrast, in the case of psoriasis, tolerance to self-nucleic acids can be breached under some pathological conditions. For example, the human antimicrobial peptide LL37 is upregulated and delivered to inflammatory sites in psoriatic skin where it forms complexes with self-nucleic acids to facilitate their entry into dendritic cells (DCs) and subsequently activate endosomal TLRs, an event that renders nonstimulatory self-nucleic acids potent immune stimuli.

Endosomal TLR3, 7, 8, and 9 are believed to be involved in a number of autoimmune diseases such as psoriasis, systemic lupus erythematous, rheumatoid arthritis, as well as cytokine storm. The development of the therapeutics to inhibit the endosomal TLRs or components of their signaling cascades is contemplated herein to provide a way to target inflammation upstream of inflammatory cytokine production. Despite the high therapeutic potential of existing drugs, their application for clinical medicine is still limited due to the lack of an appropriate drug delivery system that provides access to the TLR targets. Furthermore, the problem of implementing effective and nontoxic delivery is a severe impediment to their future therapeutic application. An effective therapeutic alternative that can be tolerated for ng periods would therefore shift the current paradigm. In accordance with the present invention, methods are provided to modulate the proinflammatory endosomal TLR-induced immune responses using engineered exosomes carrying antibodies capable of blunting their activation. Anti-TLR antibody payloads, contained within the fusion proteins and the engineered exosomes described herein, which bind the dimer-interfaces of TLR monomers, are provided in the fusion proteins described herein and the methods of using such fusion proteins and exosomes comprising them to block ligand-mediated dimerization of TLR receptor, thereby blocking pro-inflammatory signaling.

In other embodiments, the anti-TLR is fused to an antibody Fab region. In other embodiments, as set forth in Example 1.4 herein, cells can be treated with engineered exosomes comprising a tetraspanin, such as CD63 and the like, fused to defective domains, e.g., extracellular domains, and the like, of either TLR1, 2, 3, 4, 5, 6 7, 8, 9 or 10 that are defective for intracellular signaling, instead of being fused to an scFv or Fab. In particular embodiments, a dominant-negative extracellular vesicle, the tetraspanin, such as CD63, can be a fragment thereof lacking a transmembrane (e.g., 0204-224) and/or cytoplasmic domain (e.g., 0225-238)) fused to a cleavable linker (e.g., a Z-domain) fused to domains of TLR3 (lacking signal peptide and TRIF domain) or TLR7, 8, or 9 (lacking the signal peptide and TIR domains). After cleavage, the newly liberated soluble receptors undergo ligand-mediated heterodimerization with intact endosomal TLR and block pro-inflammatory signaling. Accordingly, also provided herein are dominant-negative engineered extraceluar vesicles, wherein said extracellular vesicle comprises: a tetraspanin, or fragment thereof; a cleavable linker: and a dominant-negative fragment of a TLR receptor. As used herein, the phrase “dominant-negative” (DN), in the context of TLR receptors, refers to a TLR fragment that is defective in accomplishing its natural intracellular signaling.

As used herein, the phrase “anti-TLR antibody” refers to an antibody that specifically binds to a toll-like receptor and neutralizes its effects. Particular anti-TLR antibodies, or TLR-binding fragments thereof, are antagonistic to native TLR activity. Exemplary anti-TLR antibodies, or TLR-binding fragments thereof, bind the dimer-interfaces of TLR monomers and block ligand-mediated dimerization of TLR receptor thus blocking pro-inflammatory signaling. In particular embodiments, the anti-TLR antibody specifically binds a toll-like receptor selected from the group consisting of TLR1, TLR2, TLR3, TLR4, TLR5, TLR, TLR7, TLR8,TLR9, and TLR10.

In other embodiments, the anti-TLR antibody, or functional fragment thereof, inhibits the binding of an endosomal or surface TLR to a ligand of the TLR. In other embodiments, the anti-TLR antibody, or functional fragment thereof, inhibits dimerization of an endosomal or surface TLR. In other embodiments, the anti-TLR antibody, or functional fragment thereof, inhibits endosomal or surface TLR activation via competitive or non-competitive inhibition.

The term “antibody,” or interchangeably “Ab,” is used in the broadest sense and includes fully assembled antibodies, monoclonal antibodies (including human, humanized or chimeric antibodies), polyclonal antibodies, multi-specific antibodies (e.g., bispecific antibodies), and antibody fragments that can bind antigen (e.g., Fab, Fab′, F(ab′)2, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity.

The term “monocional antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies that are antigen binding proteins are highly specific binders, being directed against an individual antigenic site or epitope, in contrast to polyclonal antibody preparations that typically include different antibodies directed against different epitopes. Non-limiting examples of monoclonal antibodies include murine, rabbit, rat, chicken, chimeric, humanized, or human antibodies, fully assembled antibodies, multi-specific antibodies (including bispecific antibodies), antibody fragments that can bind an antigen (including, Fab, Fab′, F(ab)2, Fv, single chain antibodies, diabodies), maxibodies, nanobodies, and recombinant peptides comprising CDRs of the foregoing as long as they exhibit the desired biological activity, or variants or derivatives thereof.

The modifier “monocional” indicates the character of the antibody as being obtained from a substantially homogeneous population of antibodies, and is not to be construed as requiring production of the antibody by any particular method. For example, monoclonal antibodies may be made by the hybridoma method first described by Kohler et al., Nature, 256:495 (1975), or may be made by recombinant DNA methods (see, e.g., U.S. pat. No. 4,816,567). The “monoclonal antibodies” (or “mAbs”) may also be isolated from phage antibody libraries using the techniques described in Clackson et al., Nature, 352:624-628 (1991) and Marks et al., J. Mol. Biol., 222:581-597 (1991), for example.

In an “antibody,” each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies. The constant region is the same among different antibodies. Within the variable region of each heavy or light chain, there are three hypervariable subregions that help determine the antibody's specificity for antigen in the case of an antibody that is an antigen binding protein. The variable domain residues between the hypervariable regions are called the framework residues and generally are somewhat homologous among different antibodies. Immunoglobulins can be assigned to different classes depending on the amino acid sequence of the constant domain of their heavy chains. Human light chains are classified as kappa ( [symbol] ) and lambda (λ) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)). An “antibody” also encompasses a recombinantly made antibody, and antibodies that are glycosylated or lacking glycosylation.

The term “light chain” or “immunogobulin light chain” includes a full-length light chain and fragments thereof having sufficient variable region sequence to confer binding specificity. A full-length light chain includes a variable region domain, VL and a constant region domain, CL. The variable region domain of the light chain is at the amino-terminus of the polypeptide. Light chains include kappa ( [symbol] ) chains and lambda (└) chains.

The term “heavy chain” or “immunoglobulin heavy chain” includes a full-length heavy chain and fragments thereof having sufficient variable region sequence to confer binding specificity. A full-length heavy chain includes a variable region domain, VH, and three constant region domains, CH1, CH2, and C_{H3}. The VH domain is at the amino-terminus of the polypeptide, and the CH domains are at the carboxyl-terminus, with the CH3 being closest to the carboxy-terminus of the polypeptide. Heavy chains are classified as mu (μ), delta (™), gamma (©), alpha (<), and epsilon (Σ), and define the antibody's isotype as IgM, IgD, IgG, IgA, and IgE, respectively. Heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (Fc©Rs) on the surface of immune effector cells such as natural killers and macrophages, leading to the phagocytosis or lysis of the targeted cells. In CDC, the antibodies kill the targeted cells by triggering the complement cascade at the cell surface.

An “Fc region,” or used interchangeably herein, “Fc domain” or “immunoglobulin Fc domain,” contains two heavy chain fragments, which in a full antibody comprise the Cm, and Cw domains of the antibody. The two heavy chain fragments are held together by two or more disulfide bonds and by hydrophobic interactions of the Cm domains.

The term “salvage receptor binding epitope” refers to an epitope of the Fc region of an IgG molecule (e.g., IgG1, IgG2, IgG3, IgG4) that is responsible for increasing the in vivo serum half-life of the IgG molecule.

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D. J and constant (C) gene segments are found generally in relatively close proximity on a single chromosome. During B-cell-differentiation, one of each of the appropriate family members of the V. D, J (or only V and J in the case of right chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and JH segments and may extend over as many as 10 nucleotides. Furthermore, several nucleotides may be inserted between the D and JH and between the VH and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR [i.e., residues 2434 (L1), 50-56 (12) and 89-97 (L3) in the light chain variable domain and 31-35 (H1), 5065 (H2) and 95-102 (H3) in the heavy chain variable domain as described by Kabat et al., Sequences of Proteins of Immunological Interest, 5th Ed. Public Health Service, National institutes of Health, Bethesda, Md. (1991)]. Even a single CDR may recognize and bind antigen, although with a lower affinity than the entire antigen binding site containing all of the CDRs.

An alternative definition of residues from a hypervariable “loop” is described by Chothia et al., J. Mol. Biol. 196: 901-917 (1987) as residues 26-32 (L1), 50-52 (12) and 91-96 (L3) in the light chain variable domain and 28-32 (H1), 53-55 (H2) and 96-101 (H3) in the heavy chain variable domain.

“Framework” or “FR” residues are those variable region residues other than the hypervariable region residues.

“Antibody fragments” comprise a portion of an intact full-length antibody, preferably the antigen binding or variable region of the intact antibody. Examples of antibody fragments include Fab, Fab′, F(ab′)2, and Fv fragments; diabodies; linear antibodies (Zapata et al., Protein Eng., 8(10):1057-1062 (1995)); single-chain antibody molecules; and multi-specific antibodies formed from antibody fragments.

Pepain digestion of antibodies produces two identical antigen-binding fragments, called “Fab” fragments, each with a single antigen-binding site, and a residual “Fc” fragment which contains the constant region. The Fab fragment contains all of the variable domain, as well as the constant domain of the light chain and the first constant domain (C_{H1}) of the heavy chain. The Fc fragment displays carbohydrates and is responsible for many antibody effector functions (such as binding complement and cell receptors), that distinguish one class of antibody from another.

Pepsin treatment yields an F(ab′)₂ fragment that has two “Single-chain Fv” or “scFv” antibody fragments comprising the VH and VL domains of antibody, wherein these domains are present in a single polypeptide chain. Fab fragments differ from Fab′ fragments by the inclusion of a few additional residues at the carboxy terminus of the heavy chain C_{H1} domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 113, Rosenburg and Moore eds., Springer-Vedag, New York, pp. 269-315(1994).

A “Fab fragment” is comprised of one light chain and the C_{H} and variable regions of one heavy chain. The heavy chain of a Fab molecule cannot form a disulfide bond with another heavy chain molecule.

A “Fab′ fragment” contains one light chain and a portion of one heavy chain that contains the VH domain and the C_{H1} domain and also the region between the C_{H1} and CH2 domains, such that an interchain disulfide bond can be formed between the two heavy chains of two Fab′ fragments to form an F(ab′)2 molecule.

A “F(ab′)2 fragment” contains two light chains and two heavy chains containing a portion of the constant region between the C_{H1} and C_{H2} domains, such that an interchain disulfide bond Is formed between the two heavy chains. A F(ab′)2 fragment thus is composed of two Fab′ fragments that are held together by a disulfide bond between the two heavy chains.

FV is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in international Patent Application Publication No. WO 88/01649 and U.S. Pat. Nos. 4,946,778 and 5,260,203, the disclosures of which are incorporated by reference in their entireties.

“Single-chain Fv” or “scFv” antibody fragments comprise the VH and VL domains of antibody, wherein these domains are present in a single polypeptide chain, and optionally comprising a polypeptide linker between the VH and VL domains that enables the Fv to form the desired structure for antigen binding (Bird et al., Science 242:423-426, 1988, and Huston et al., Proc. Natl. Acad. Sci. USA 85:5879-5883, 1988). An “Fd” fragment consists of the VH and CH, domains.

The term “diabodies” refers to small antibody fragments with two antigen-binding sites, which fragments comprise a heavy-chain variable domain (VH) connected to alight-chain variable domain (VL) in the same polypeptide chain (VH VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of alight chain. In some instances, two or more VH regions are covalently joined with a peptide linker to create a bivalent domain antibody. The two VH regions of a bivalent domain antibody may target the same or different antigens.

In general, an antibody, or antibody fragment, “specifically binds” to an antigen of interest when it has a significantly higher binding affinity for, and consequently is capable of distinguishing, that antigen, compared to its affinity for other unrelated proteins, under similar binding assay conditions. Typically, an antibody is said to “specifically bind” its target antigen when the dissociation constant (KD) is in the range of about 500 nM-10⁻⁸ M, or lower. The antibody specifically binds antigen with “high affinity” when the KD is 10⁻⁹ M or lower, and with “very high affinity” when the KD is 10⁻¹⁰ M or lower.

“Antigen binding region” or “antigen binding site” means a portion of a protein that specifically binds a specified antigen. For example, that portion of an antibody, or antibody fragment, that contains the amino acid residues that interact with an antigen and confer on the antigen binding protein its specificity and affinity for the antigen is referred to as “antigen binding region.” An antigen binding region typically includes one or more “complementary binding regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen. In a traditional antibody, the CDRs are embedded within a framework in the heavy and light chain variable region where they constitute the regions responsible for antigen binding and recognition. A variable region of an immunoglobulin antigen binding protein comprises at least three heavy or light chain CDRs, see, supra (Kabat et al., 1991, Sequences of Proteins of Immunological Interest. Public Health Service N.I.H., Bethesda, Md.; see also Chothia and Lesk, 1987, J. Mol. Biol. 196:901917; Chothia et al., 1989, Nature 342: 877-883), within a framework region (designated framework regions 1-4, FR1. FR2, FR3, and FR4, by Kabat et al., 1991, supra; see also Chothia and Lesk, 1987, supra).

The term “antigen” refers to a molecule or a portion of a molecule capable of being bound by a selective binding agent (e.g., an antibody or immunologically functional fragment of an antibody), and additionally capable of being used in an animal to produce antibodies capable of binding to that antigen. An antigen may possess one or more epitopes that are capable of interacting with different antibodies.

The term “epitope” is the portion of a molecule that is bound by an antibody (or antibody fragment). The term includes any determinant capable of specifically binding to an antibody (or antibody fragment), or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three-dimensional structure that is similar to an epitope used to generate the antigen binding protein, yet they also comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three-dimensional structural characteristics, and/or specific charge characteristics. Generally, antibodies specific for a particular target antigen will preferentially recognize an epitope on the target antigen in a complex mixture of proteins and/or macromolecules.

Pharmaceutical Compositions

Also provided herein are pharmaceutical compositions comprising an engineered extracellular vesicle, such as an engineered exosome as described herein, and a pharmaceutically acceptable carrier or excipient. Also provided herein, are methods of treating, preventing, ameliorating and/or inhibiting an inflammatory response in an individual in need thereof, the method comprising administering to the individual therapeutically effective amount of a pharmaceutical composition of the invention. The inflammatory response can be a symptom of viral infection, autoimmune disease or chronic immune activation. In some embodiments, the extracellular vesicle is selected from an exosome, ectosome or microvesicle. In a particular embodiment, the extracellular vesicle is an engineered exosome.

As used herein, a pharmaceutical composition refers to any mixture comprising an engineered extracellular vesicle provided herein, such as an engineered exosome. It can be a solution, a suspension, liquid, powder, a paste, aqueous or non-aqueous formulations or any combination thereof.

Pharmaceutical compositions containing the engineered extracellular vesicles described herein, either as separate agents or in combination in a composition mixture can be formulated in any conventional manner by mixing a selected amount of the respective extracellular vesicle, such as an engineered exosome, with one or more physiologically acceptable carriers or excipients. Selection of the carrier or excipient is within the skill of the administering profession and can depend upon a number of parameters. These include, for example, the mode of administration (i.e., systemic, oral, nasal, pulmonary, local, topical, or any other mode) and disorder treated. The pharmaceutical compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration. To formulate a composition, the weight fraction of an EV, e.g., and exosome, or mixture thereof is dissolved, suspended, dispersed, or otherwise mixed in a selected vehicle at an effective concentration such that the treated condition is relieved or ameliorated.

The terms “administering” and “administration” refer to methods of providing a pharmaceutical preparation to a subject. Such methods are well known to those skilled in the art and Include, but are not limited to, administering the compositions orally, intranasally, intratracheally, eye drops, intrathecal, parenterally (e.g., intravenously and subcutaneously), by intramuscular injection, by intraperitoneal injection, intrathecally, transdermally, extracorporeally, topically or the like.

Generally, pharmaceutically acceptable compositions are prepared in view of approvals for a regulatory agency or other prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions.

It is understood that appropriate doses depend upon a number of factors within the level of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the therapeutic agent to have upon the subject. Exemplary doses include milligram or microgram amounts of the therapeutic agent per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency. Such appropriate doses may be determined using the assays known in the art. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, and any drug combination.

Parenteral compositions may be formulated in dosage unit form for ease of administration and uniformity of dosage. Dosage unit form as used herein refers to physically discrete units suited as unitary dosages for the subjects to be treated; each unit containing a predetermined quantity of compound of the invention calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a compound of the invention for the treatment of the disease.

Methods of Treatment:

In certain embodiments of the methods described herein, the composition is suitable for treating, preventing, ameliorating and/or inhibiting a disease or malady selected from the group of: cytokine storm, Systemic Lupus Erythematous (SLE), psoriasis, eczema, seborrheic dermatitis, actinic keratosis, glomerulonephritis, SjOgren's syndrome, systemic inflammatory syndrome (SIRS, e.g. sepsis with and without documented pathogen), macrophage activation syndrome (MAS), severe acute respiratory syndrome (SARS), hantavirus pulmonary syndrome, disseminated vascular coagulopathy (DIC), glomerulonephritis, diabetes (Type 1 and 2), traumatic brain injury, transplant, graft vs. host disease, liver fibrosis, pulmonary fibrosis, chronic obstructive pulmonary disease, asthma, influenza, human immunodeficiency virus (HIV) infection, hepatitis infection, viral pneumonia, rheumatoid arthritis, acute lung injury, crush injury, traumatic injury, bone fracture, metabolic syndrome, atherosclerosis, Addison's Disease, pheochromocytoma, metastasis, hyperacute insect sting, anaphylaxis, necrosis associated with flesh-eating bacterial infection, kidney fibrosis, lupus nephritis, radiation therapy, frostbite, ischemia, reperfusion, myocardial infarction, myocarditis, bacterial pneumonia, bacterial sepsis, COVID-19 and Legionnaires disease. In other embodiments, methods are provided for treating, preventing, alleviating, stabilizing the inflammatory response mediated by endosomal TLR activation. Also provided herein, is a method of treating, preventing or delaying the onset of cytokine storm in an individual suspected or at risk of developing septic shock, the method comprising administering to the individual a prophylactically effective amount of a pharmaceutical composition of the invention.

As used herein, the phrase “cytokine storm” refers to an excessively activated cytokine cascade or hypercytokinemia, i.e., an excessive or uncontrolled release of proinflammatory cytokines, which can be associated with a wide variety of infectious and noninfectious diseases or disorders. Cytokine storm syndromes are a group of disorders (such as, but not limited to, influenza, asthma, hantavirus pulmonary syndrome. SIRS, MAS, SARS, COVID-19 and DIC, as described above), representing a variety of inflammatory causes; the present invention does not depend on any particular one of these underlying causes, but is directed to preventing or delaying the onset of cytokine storm, or treating cytokine storm, arising from any underlying inflammatory cause. Typically, the primary symptoms of a cytokine storm are high fever, swelling and redness, extreme fatigue and nausea. In some cases, the immune reaction can result in bleeding, clotting, internal organ injury, or shock, and may be fatal.

As used herein, the phrase “inflammatory response” or “pro-inflammatory response” or grammatical variations thereof, refers to an immune response by an animal or human body to infection, injury, or to autoimmunity or chronic immune activation, which response is typically characterized by pain, heat (localized warmth or fever), redness, swelling, and/or loss of normal function, in alocalized area of the body or more generally. Included in “inflammatory response” is acute inflammation, which lasts only a few days, and chronic inflammation, which can last weeks, months, or years, and may be relapsing.

The terms “treatment” or “treating” of a subject includes the application or administration of an effective amount of a pharmaceutical composition of the invention to a subject (or application or administration of a compound or pharmaceutical composition of the invention to a cell or tissue from a subject) with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; lessening of the rate of worsening; stabilization, diminishing of symptoms or making the injury, pathology or condition more tolerable to the subject; slowing in the rate of degeneration or decline; making the final point of degeneration less debilitating; or improving a subject's physical or mental well-being. In an embodiment, the term “treating” can include increasing a subjec's life expectancy.

As used herein an “effective amount” of a compound or composition for treating a particular disease, such as those caused by an untoward inflammatory response, including cytokine storm, and the like, is an amount that is sufficient to ameliorate, or In some manner reduce the symptoms associated with the disease. Such amount can be administered as a single dosage or can be administered according to a regimen, whereby it is effective. The amount can cure the disease but, in certain embodiments, is administered in order to ameliorate the symptoms of the disease. In particular embodiments, repeated administration is required to achieve a desired amelioration of symptoms. A “therapeutically effective amount” or “herapeutically effective dose” can refer to an agent, compound, material, or composition containing a compound, such as an engineered extracellular vesicle as described herein, that is at least sufficient to produce a therapeutic effect. An effective amount is the quantity of a therapeutic agent necessary for preventing, curing, ameliorating, arresting or partially arresting a symptom of a disease or disorder.

As used herein, ‘patient or’subject to be treated includes humans and or non-human animals, including mammals. Mammals include primates, such as humans, chimpanzees, gorillas and monkeys; and domesticated animals.

Also provided is a method for delivering a therapeutic molecule to an individual in need thereof, the method comprising: (1) harvesting exosomes from a cultured packaging cell line, wherein the packaging cell line comprises an expression vector encoding a fusion protein: and (2) administering an engineered exosomes harvested in step (1) to an individual in need thereof.

Also provided herein, is an exosome-based delivery system for delivering a therapeutic molecule, wherein said delivery system comprises a fusion protein comprising an anti-TLR antibody fused with a tetraspanin, wherein said tetraspanin is positioned at the surface of an exosome, and wherein said tetraspanin is capable of binding to a target on recipient cell. The tetraspanin member of the tetraspanin family of proteins is selected from the group consisting of: TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 18), TSPAN21(uroplakin1A) TSPAN22 (peripherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, and TSPAN34.

Fusion Proteins:

Also provided, is a fusion protein comprising a tetraspanin, or a functional fragment thereof, linked to an anti-TLR antibody, or a functional fragment thereof, wherein the anti-TLR antibody inhibits an endosomal TLR activity. In particular embodiments, the tetraspanin is selected from the group consisting of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 1B), TSPAN21 (uroplakin1A) TSPAN22 (perpherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, TSPAN34; and the anti-TLR antibody specifically binds a toll-like receptor selected from the group consisting of TLR2, TLR3, TLR4, TLR5, TLR6, TLR7, TLR8, and TLR9. In some embodiments, the tetraspanin is TSPAN30 (CD63). In some embodiments, the anti-TLR antibody, or functional fragment thereof, inhibits the binding of an endosomal TLR to a ligand of the TLR. In other embodiments, the anti-TLR antibody, or functional fragment thereof, inhibits dimerization of an endosomal TLR. In particular embodiments, the fusion protein comprises a peptidyl linker comprising a cleavage site of protease/endopeptidase. In certain embodiments, the cleavage site is recognized by a protease/endopeptidase selected from the group consisting of cathepsin B, cathepsin H, cathepsin K, cathepsin L, cathepsin S, asparagine endopeptidase, and furin-like protease.

In some embodiments, the fusion proteins can be selected from the group of fusion proteins set forth in Table 3 that have an anti-TLR antibody, or a functional fragment thereof therein. In other embodiments, the fusion protein can comprise a tetraspanin, or a functional fragment thereof, linked to a functional moiety, wherein said fusion protein is selected from the group of fusion proteins set forth in Table 3.

The term “fusion protein,” indicates that the protein includes polypeptide components derived from more than one parental protein or polypeptide, for example, a tetraspanin (or a functional fragment thereof) and an anti-TLR antibody (or functional fragment thereof). Typically, a fusion protein is expressed from a “fusion gene” in which a nucleotide sequence encoding a polypeptide sequence from one protein is appended in frame with (optionally separated by a gene sequence encoding a peptidyl linker) a nucleotide sequence encoding a polypeptide sequence from a different protein. The fusion gene can then be expressed by a recombinant host cell as a single protein.

“Polypeptide” and “protein” are used interchangeably herein and include a molecular chain of two or more amino acids linked covalently through peptide bonds. The terms do not refer to a specific length of the product. Thus. “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are Included within the meaning of polypeptide. The terms also include molecules in which one or more amino acid analogs or non-canonical or unnatural amino acids are included as can be expressed recombinantly using known protein engineering techniques. In addition, fusion proteins can be derivatized as described herein by well-known organic chemistry techniques.

A “variant” of a polypeptide (e.g., an immunoglobulin, or an antibody) comprises an amino acid sequence wherein one or more amino acid residues are inserted into, deleted from and/or substituted into the amino acid sequence relative to another polypeptide sequence. Variants include variant fusion proteins.

The fusion protein for use in accordance with the invention is typically produced by recombinant expression technology. The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., duing cloning, DNA shuffling or other well-known molecular biological procedures. Examples of such molecular biological procedures are found in Maniatis et al., Molecular Cloning. A Laboratory Manual. Cold Spring Harbor Laboratory, Cold Spring Harbor. N.Y. (1982). A “recombinant DNA molecule,” is comprised of segments of DNA joined together by means of such molecular biological techniques.

Cloning of DNA is carried out using standard techniques (see, e.g., Sambrook et al. (1989) Molecular Cloning: A Laboratory Guide, Vols 1-3, Cold Spring Harbor Press, which Is incorporated herein by reference). For example, a cDNA library may be constructed by reverse transcription of polyA+mRNA, preferably membrane-associated mRNA, and the library screened using probes specific for human immunoglobulin polypeptide gene sequences. In some embodiments, however, the polymerase chain reaction (PCR) is used to amplify cDNAs (or portions of full-length cDNAs) encoding an immunoglobulin gene segment of interest (e.g., alight or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the polypeptide of interest, e.g., a portion of the fusion protein sequence.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

Sequencing of DNA is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Aced. Sci. USA 74: 5463-5467, which is incorporated herein by reference). By comparing the sequence of the cloned nucleic acid with published sequences of genes and cDNAs, one of skill will readily be able to determine, depending on the region sequenced. One source of gene sequence information is the National Center for Biotechnology Information, National Library of Medicine, National Institutes of Health, Bethesda, Md.

The term “identity” of a sequence refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje. G., 1987. Sequence Analysis in Molecular Biology, New York: Academic Press; Sequence Analysis Primer, (Gribskov, M. and Devereux, J., eds.), 1991, New York: M. Stockton Press: and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned or optimal matching of their respective residues (either along the full length of one or both sequences, or along a pre-determined portion of one or both sequences). The programs provide a default opening penalty and a default gap penalty, and a scoring matrix such as PAM 250 [a standard scoring matrix: see Dayhoff et al., in Atlas of Protein Sequence and Structure, vol. 5. supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12; 387; Genetics Computer Group, University of Wisconsin, Madison, Wis.). The computer algorithm GAP Is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3×the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm

Recommended parameters for determining percent identity for polypeptides or nucleotide sequences using the GAP program include the following: Algorithm: Needleman et al., 1970, J. Mol. Bio. 48:443-453; Comparison matrix: BLOSUM 82 from Henikoff et al., 1992, supra; Gap Penalty: 12 (but with no penalty for end gaps) Gap Length Penalty: 4 Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with proteins of interest, include, but are not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. By methods known to the skilled artisan, proteins, can be engineered or modified for Improved target affinity, selectivity, stability, and/or manufacturability before the coding sequence of the engineered protein is included in the expression cassette.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the expressed protein; an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are known in the art.

The term “recombinant protein” or “recombinant polypeptide.” as used herein, refers to a protein molecule which is expressed using a recombinant DNA molecule. A “recombinant host cell” is a cell that contains and/or expresses a recombinant nucleic acid. Recombinant DNA molecules useful in expressing the fusion protein are described, e.g., by Papadopoulos et al., Modified Chimeric Polypeptides with Improved Pharmacokinetic Properties, U.S. Pat. No. 7,070,959 B2; and WO 00/75319 A1).

The term “naturally occurring,” where it occurs in the specification in connection with biological materials such as polypeptides, nucleic acids, host cells, and the like, refers to materials which are found in nature.

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review, see Voss, et al., Trends Biochem. Sci., 11:287 (1986) and Maniatis, et al., Science 236:1237 (1987)).

A “promoter” is a region of DNA including a site at which RNA polymerase binds to initiate transcription of messenger RNA by one or more downstream structural genes. Promoters are located near the transcription start sites of genes, on the same strand and upstream on the DNA (towards the 5′ region of the sense strand). Promoters are typically about 100-1000 bp in length.

An “enhancer” is a short (50-1500 bp) region of DNA that can be bound with one or more activator proteins (transcription factors) to activate transcription of a gene.

The terms “in operable combination,” “in operable order.” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

A “secreted” protein refers to those proteins capable of being directed to the endoplasmic reticulum (ER), secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments, the fusion protein of interest can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion, or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing an ionic detergent or chaotropic agent, such as sodium dodecyl sulfate (SDS), urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc., or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing an ionic detergent or chaotropic agent, such as sodium dodecyl sulfate (SDS), urea, guanidinium hydrochloride, or lithium perchlorate.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein Is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it Is found in nature. However, an isolated nucleic acid molecule includes a nucleic acid molecule contained in cells that ordinarily express the immunoglobulin (e.g., antibody) where, for example, the nucleic acid molecule is in a chromosomal location different from that of natural cells.

As used herein, the terms nucleic acid molecule encoding, “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly Including subsequent post-translational modification of the polypeptide), or both transcription and translation, as indicated by the context.

An expression cassette is atypical feature of recombinant expression technology. The expression cassette includes a gene encoding a protein of interest e.g., a gene encoding the fusion protein sequence. A eukaryotic “expression cassette” refers to the part of an expression vector that enables production of protein in a eukaryotic cell, such as a mammalian cell. It includes a promoter, operable in a eukaryotic cell, for mRNA transcription, one or more gene(s) encoding protein(s) of interest and a mRNA termination and processing signal. An expression cassette can usefully include among the coding sequences, a gene useful as a selective marker. In the expression cassette promoter is operably linked 5′ to an open reading frame encoding an exogenous protein of interest; and a polyadenylation site is operably linked 3′ to the open reading frame. Other suitable control sequences can also be included as long as the expression cassette remains operable. The open reading frame can optionally include a coding sequence for more than one protein of interest.

As used herein the term “coding region” or “coding sequence” when used in reference to a structural gene refers to the nucleotide sequences which encode the amino acids found in the nascent polypeptide as a result of translation of an mRNA molecule.

The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Recombinant expression technology typically involves the use of a recombinant expression vector comprising an expression cassette and a mammalian host cell comprising the recombinant expression vector with the expression cassette or at least the expression cassette, which may for example, be integrated into the host cell genome.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (See, e.g., Goodey, Andrew R.; et al., Peptide and DNA sequences, U.S. Pat. No. 5,302,697: Weiner at al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1). For expression of multi-subunit proteins of interest, separate expression vectors in suitable numbers and proportions, each containing a coding sequence for each of the different subunit monomers, can be used to transform a host cell. In other embodiments, a single expression vector can be used to express the different subunits of a protein of interest.

The term “host cell” means a cell that has been transformed, or is capable of being transformed, with a nucleic acid and thereby expresses a gene or coding sequence of interest.

The term includes the progeny of the parent cell, whether or not the progeny is identical in morphology or in genetic make-up to the original parent cell, so long as the gene of interest is present. Any of a large number of available and well-known host cells may be used in the practice of this invention to obtain the fusion protein, although mammalian host cells capable of post-translational glycosylation may be preferred for some embodiments. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Modifications can be made at the DNA level, as well. The peptide-encoding DNA sequence may be changed to codons more compatible with the chosen host cell. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

Within these general guidelines, microbial host cells in culture, such as bacteria (such as Escheilchia coli sp.), and yeast cell lines (e.g., Saccharomyes, Pichia, Schizosaccharomyces, Kluyveromyces) and other fungal cells, algal or algal-like cells, insect cells, plant cells, that have been modified to incorporate humanized glycosylation pathways, can also be used to produce functional glycosylated fusion protein, if desired. However, mammalian (including human) host cells, e.g., CHO cells and HEK-293 cells, are particularly useful.

Examples of useful mammalian host cell lines are Chinese hamster ovary cells, including CHO-K1 cells (e.g., ATCC CCL61), DXB-11, DG-44, and Chinese hamster ovary cells/-DHFR (CHO, Udaub et al, Proc. Natl. Acad. Sci. USA 77: 4216 (1980)); monkey kidney CVI line transformed by SV40 (COS-7, ATCC CRL 1651); human embryonic kidney line (293 or 293 cells subcloned for growth in suspension culture (Graham et al, J. Gen Viol. 36: 59 (1977)); baby hamster kidney cells (BHK, ATCC CCL 10); mouse Sertoli cells (TM4, Mather, Bid. Reprod. 23: 243-251 (1980)); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383:44-68 (1982)); MRC 5 cells or FS4 cells; or mammalian myeloma cells, e.g., NS0 or sp2/0 mouse myeloma cells.

“Cell,” “cell line,” and “cell culture” are often used interchangeably and all such designations herein include cellular progeny. For example, a cell “derived” from a CHO cell is a cellular progeny of a Chinese Hamster Ovary cell, which may be removed from the original primary cell parent by any number of generations, and which can also include a transformant progeny cel.

Transformants and transformed cells include the primary subject cell and cultures derived therefrom without regard for the number of transfers. It Is also understood that all progeny may not be precisely identical in DNA content, due to deliberate or inadvertent mutations. Mutant progeny that have the same function or biological activity as screened for in the originally transformed cell are included.

Host cells are transformed or transected with the above-described nucleic acids or vectors for production of polypeptides (including antigen binding proteins, such as antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host calls.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically Integrating into a chromosome of the cell, or may be maintained transiently as an episomal element without being replicated, or may replicate independently as a plasmid. A cell is considered to have been “stably transformed” when the transforming DNA is replicated with the division of the cell.

The term “linked” or “linker,” in the context of the tetraspanin and anti-TLR antibody complex of the invention, refers to any mechanistic, chemical, or recombinant approach, such as using a fusion protein, for attaching a polypeptide having TLR-specific binding activity under physiological conditions with a tetraspanin peptide, polypeptide, or protein. As used herein, the phrase “fused” or “fusion,” or grammatical variations thereof, refers to two or more polypeptide moieties or domains that are covalently linked, directly or indirectly (e.g., via an intervening amino acid sequence), which polypeptide moieties or domains do not naturally occur in the same molecule. The “fusion” of the second polypeptide moiety to the first polypeptide moiety can be a direct fusion of the sequences, with the second peptide directly adjacent to the first peptide, or it may be an indirect fusion, e.g., with intervening amino acid sequence such as a cleavable linker, an identifier or epitope tag sequence, a domain, a functional peptide, or a larger polypeptide or protein. In some embodiments, the fused polypeptides are expressed from a gene that codes for both of them so that upon expression of the gene, the polypeptides are part of the same protein (e.g., a fusion protein). In other embodiments, the two peptides may be linked following co-expression in a recombinant host cell, using high affinity binding sequences between the two peptides, such as biotin and avidin or strepavidin. In yet other examples, the two peptides are linked following expression and purification of each polypeptide, after which they are synthetically tethered together, perhaps by linking the C-terminus of the first polypeptide to the N-terminus of the other polypeptide.

In certain embodiments, the anti-TLR antibody (or functional fragment thereof) is fused to the tetraspanin (or functional fragment thereof), with a linker that is susceptible to cleavage by proteases/endopeptidases activated in an acidic environment in the endosome, yielding free payload in the endosomal compartment. Such proteases/endopeptidases include, but are not limited to cathepsin, cathepsin H, cathepsin K, cathepsin L, or cathepsin S; or other endopeptidases e.g. asparagine endopeptidase. Amino acid residues constituting cleavage target sites of such proteases or endopeptidases are known in the art and can be incorporated into the linker sequence employed. (See, e.g., Biniossek et al., Proteomic Identification of Protease Cleavage Sites Characterizes Prime and Non-prime Specificity of Cysteine Cathepsins B, L, and S, J. Proteome Res., 2011, 10 (12), pp 5363-6373 (2011) and Erratum in J. Proteome Res. 10(12):5577 (2011); Manoury et al., An asparaginyl endopeptidase processes a microbial antigen for class II MHC presentation, Nature 396: 695-699 (1998): Maschalidi et al., Asperagine Endopeptidase Controls Anti-Influenza Virus Immune Responses through TLR7 Activation, PLoS Pathogens 8(8):e1002841. doi.oro/10.1371Coumalowt1002841(2012); Sun et al., Proteolytic Characteristics of Cathepsin D Related to the Recognition and Cleavage of its Target Proteins, PLoS One 8(6): e65733. doi.oro/10.1371mioumal.one.0065733: Gikanga et al., Cathepsin B Cleavage of vcMMAE-Bsed Anhbody-Drug Conjugate s Not Drug Location or Monoclonal Antibody Carrier Specific, Bloconjugate Chem., 2016, 27 (4):1040-104 (2016); Yasothomsikul et al., Cathepsin L in secretory vesicles functions as a prohormone-processing enzyme for production of the enkephalin peptide neurotransmitter, Proc. Natl. Acad. Sci. (USA) 100 (16):9590-9595 (2003)).

In other embodiments, the linker's chemical structure is not critical, since it serves primarily as a spacer to position, join, connect, or optimize presentation or position of one functional moiety in relation to one or more other functional moieties of a molecule. The presence of a linker moiety can be useful in optimizing pharmacological activity of some embodiments of the invention. The linker is preferably made up of amino acids linked together by peptide bonds. The linker moiety, if present, can be independently the same or different from any other linker, or linkers, that may also be present in the fusion protein.

As stated above, the linker moiety, if present, can be “peptidyl” in nature (i.e., made up of amino acids linked together by peptide bonds) and made up in length, preferably, of from 1 up to about 40 amino acid residues, more preferably, of from 1 up to about 20 amino acid residues, and most preferably of from 1 to about 10 amino acid residues. Preferably, but not necessarily, the amino acid residues in the linker are from among the twenty canonical amino acids, more preferably, cysteine, glycine, alanine, proline, asparagine, glutamine, and/or serine. Even more preferably, a peptidyl linker Is made up of a majority of amino acids that are sterically unhindered, such as glycine, serine, and alanine linked by a peptide bond. It is also desirable that, if present, a peptidyl linker be selected that avoids rapid proteoytic turnover in circulation in vivo. Some of these amino acids may be glycosylated, as is well understood by those in the art. For example, a useful linker sequence constituting a sialylation site is X₁X₂NX₄X₅G, wherein X₁, X₂, X₄ and X₅ are each independently any amino acid residue.

In other embodiments, the 1 to 40 amino acids of the peptidy linker moiety are selected from glycine, alanine, proline, asparagine, glutamine, and lysine. Preferably, a linker is made up of a majority of amino acids that are satirically unhindered, such as glycine and alanine. Thus, preferred linkers include polyglycines, polyserines, and polyalanines, or combinations of any of these. Some exemplary peptidy linkers are poly(Gly)₁₋₈, particularly (Gly)3, (Gly)4, (Gly)5 and (Gly)7. Other specific examples of peptidy linkers include (Gly)5Lys, and (Gly)LysArg. Other examples of useful peptidyl linkers are: (Gly)3Lys(Gly)4; (Gly)3AsnGlySer(Giy)2; (Gly)3Cys(Gly)4; and GlyProAsnGlyGly. To explain the above nomenclature, for example, (Gly)3Lys(Gly)4 means Gly-Gly-Gly-Lys-Gly-Gly-Gly-Gly. Other combinations of Gly and Ala are also useful. Commonly used peptidyl linkers include GGGGS (SEQ ID NO:185); GGGGSGGGGS (SEQ ID NO: 186); GGGGSGGGGSGGGGSGGGGSGGGGS (SEQ ID NO: 187) and any linkers used in the working examples hereinafter.

In some embodiments of the compositions of this invention, which comprise a peptide linker moiety, acidic residues, for example, glutamate or aspartate residues, are placed in the amino acid sequence of the linker moiety.

In some embodiments of the compositions of this invention, which comprise a peptide or peptidyl linker moiety, acidic residues, for example, glutamate or aspartate residues, are placed in the amino acid sequence of the linker moiety. Examples include the following peptide linker sequences: GGEGGG (SEQ ID NO: 162); GGEEEGGG (SEQ ID NO: 163); GEEEG (SEQ ID NO: 164); GEEE (SEQ ID NO: 165); GGDGGG (SEQ ID NO: 166): GGDDDGG (SEQ ID NO: 167): GDDD (SEQ ID NO: 168); GDDD (SEQ ID NO: 169); GGGGSDDSDEGSDGEDGGGGS (SEQ ID NO: 170): WEWEW (SEQ ID NO: 171); FEFEF (SEQ ID NO: 172); EEEWWW (SEQ ID NO: 173); EEEFFF (SEQ ID NO: 174); WWEEEWW (SEQ ID NO: 175); or FFEEEFF (SEQ ID NO: 176).

In other embodiments, the linker constitutes a phosphorylation site, e.g., X1X2YX4X5G, wherein X1, X2, X4, and X5 are each independently any amino acid residue; X1X2SX4X5G, wherein X1, X2, X4 and X5 are each independently any amino acid residue; or X₁X₂TX₄X₅G, wherein X1, X2, X4 and X5 are each independently any amino acid residue.

The linkers shown here are exemplary; peptidyl linkers within the scope of this invention may be much longer and may include other residues. A peptidyl linker can contain, e.g., a cysteine, another thiol, or nucleophile for conjugation with a half-life extending moiety. In other embodiments, the linker contains a cysteine or homocysteine residue, or other 2-amino-ethanethiol or 3-amino-propanethiol moiety for conjugation to maleimide, iodoacetamide or thioester, functionalized half-life extending moiety.

Another useful peptidyl linker is a large, flexible linker comprising a random Gly/Ser/Thr sequence, for example: GSGSATGGSGSTASSGSGSATH (SEQ ID NO: 177) or HGSGSATGGSGSTASSGSGSAT (SEQ ID NO: 178), that is estimated to be about the size of a 1 kDa PEG molecule. Alternatively, a useful peptidyl linker may be comprised of amino acid sequences known in the art to form rigid helical structures (e.g., Rigid linker -AEAAAKEAAAKEAAAKAGG-(SEQ ID NO: 179)). Additionally, a peptidyl linker can also comprise a non-peptidyl segment such as a 6-carbon aliphatic molecule of the formula -CH2-CH2-CH2-CH2-CH2-CH2-. The peptidyl linkers can be altered to form derivatives as described herein.

Optionally, a non-peptidyl linker moiety Is also useful for conjugating the tetraspanin, or functional fragment thereof, to the anti-TLR antibody, or functional fragment thereof. For example, alkyl linkers such as —NH—(CH2)S—C(O)— wherein s=2-20 can be used. These alkyl linkers may further be substituted by any non-sterically hindering group such as lower alkyl (e.g., C1-C6)lower acyl, halogen (e.g., Cl, Br), CN, NH2, phenyl, etc. Exemplary non-peptidyl linkers are polyethylene glycol (PEG) linkers having a molecular weight of about 100 to about 5000 Daltons (Da), preferably about 100 to about 500 Da.

In some embodiments, the non-peptidyl linker is aryl. The linkers may be altered to form derivatives in the same manner as described in the art, e.g., in Sullivan et al, Toxin Peptide Therapeudc Agents, US2007/0071764; Sullivan et al, Toxin Peptide Therapeutic Agents, PCT/US2007/022831, published as WO2008/088422, which are all incorporated herein by reference in their entireties. “Aryl” is phenyl or phenyl vicinally-fused with a saturated, partially-saturated, or unsaturated 3-, 4-, or 5-membered carbon bridge, the phenyl or bridge being substituted by 0, 1, 2 or 3 substituents selected from C₁₋₈ alkyl, C1-4 haloalkyl or halo. “Heteroaryl” is an unsaturated 5, 6 or 7 membered monocyclic or partially-saturated or unsaturated 6-, 7-, 8-, 9-, 10- or 11 membered bicyclic ring, wherein at least one ring is unsaturated, the monocyclic and the bicyclic rings containing 1, 2, 3 or 4 atoms selected from N, O and S, wherein the ring is substituted by 0, 1, 2 or 3 substituents selected from C₁₋₆ alkyl, C₁₋₄ haloalkyl and halo.

Non-peptide portions of the inventive composition of matter, such as non-peptidyl linkers or non-peptide half-life extending moieties can be synthesized by conventional organic chemistry reactions.

The above is merely illustrative and not an exhaustive treatment of the kinds of linkers that can optionally be employed in accordance with the present invention.

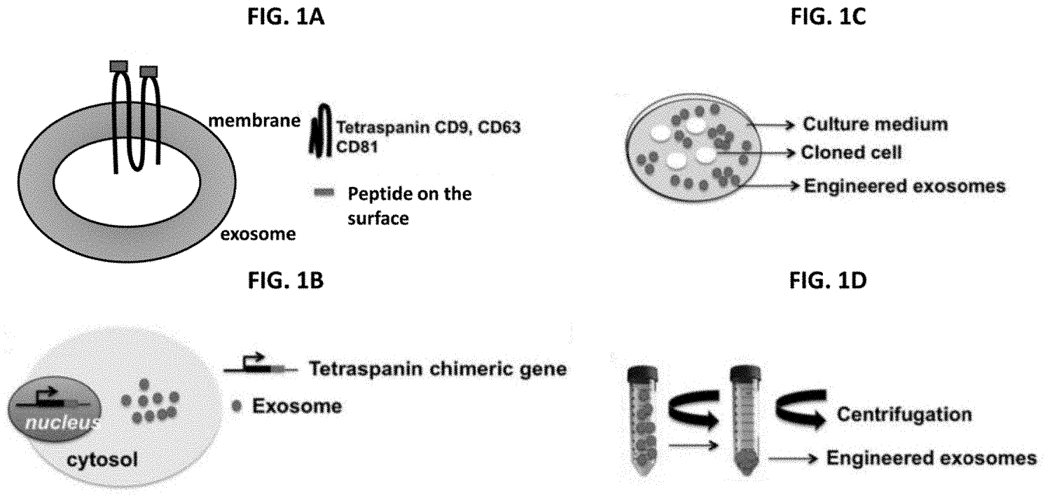

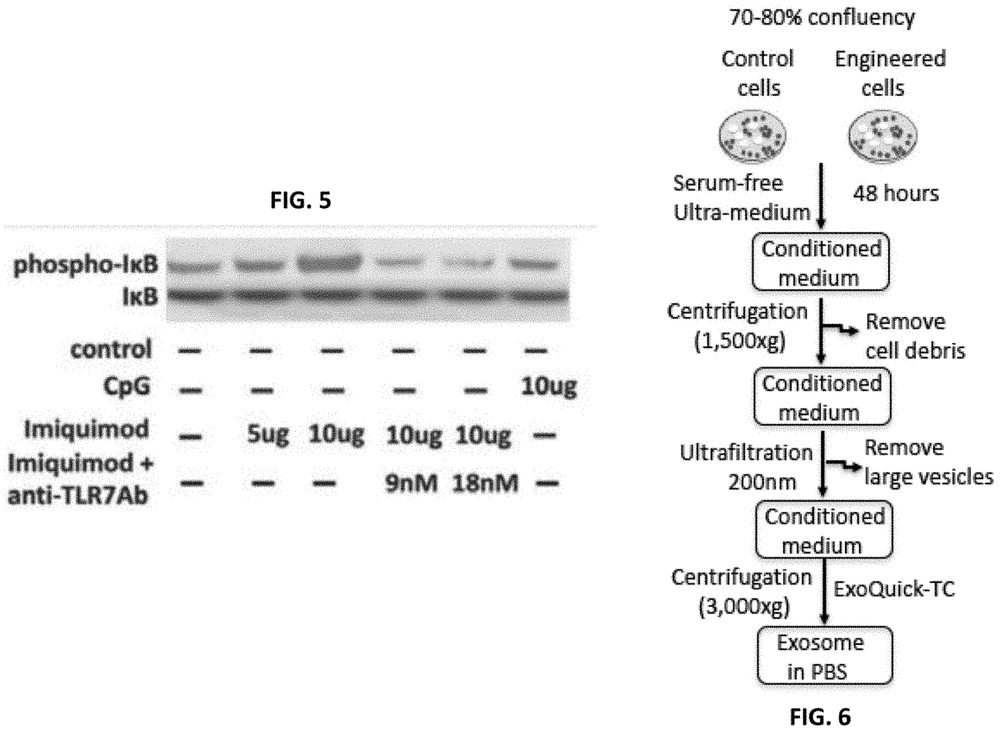

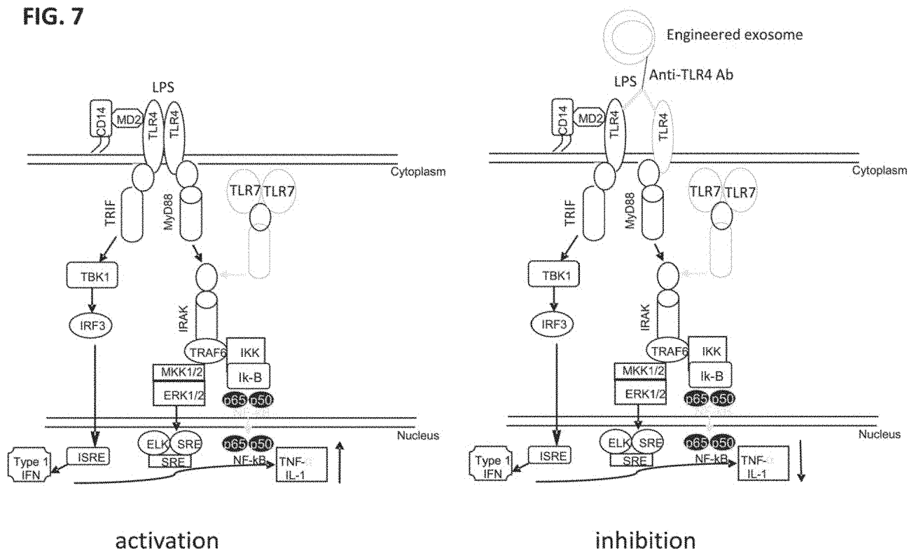

Referring now to FIGS. 1A-7, the present invention features an engineered extracellular vesicle, preferably an exosome, comprising a fusion protein having tetraspanin, or functional fragment thereof, linked to anti-TLR antibody, or functional fragment thereof, that inhibits cell surface expressed TLRs as well are endosomal TLRs.

In some embodiments, the present invention may feature an engineered extracellular vesicle comprising a fusion protein having tetraspanin, or a functional fragment thereof, linked to an anti-TLR antibody, or a functional fragment thereof, that inhibits cell surface expressed TLRs as well as an endosomal TLRs.

In some embodiments, the present invention features an engineered exosome that display on the exosome surface or inside the exosome a fusion protein comprising a tetraspanin, such as CD63, fused to an anti-TLR antibody that target and inhibits cell surface expressed TLRs and may inhibit endosomal TLRs (e.g., TLR3, TLR7, TLR8, TLR9, TLR2, TLR4, TLR5, and TLR6-mediated cytokine storms. In some embodiments, the anti-TLR antibody inhibits activation of TLRs binding to their respective ligands. In other embodiments, the anti-TLR antibody inhibits activation of dimerization of endosomal TLRs, which is required for activation. In other embodiments, anti-TLR antibody inhibits endosomal TLR activation via competitive or non-competitive inhibition.

In some embodiments, the extracellular vesicle is an exosome. Other extracellular vesicles may include, but are not limited to, microvesicles (150 nm-1(m) and apoptotic bodies (>1(m). In some embodiments, apoptotic bodies drive a proinflammatory responses.

In particular embodiments, the antibody payload is linked or fused to the tetraspanin via one or more protease/endopeptidase recognition sequences cleavable by protease/endopeptidase, such as cathepsins B, H, K, L, and S, as well as aspargine endopeptidase, furin-like proteases, and the like, which are enzymatically active when the exosomes reach the acidic endosomal environment.

In some embodiments, the engineered extracellular vesicle comprises a tetraspanin selected from the group consisting of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 1B), TSPAN21 (uropakin1A) TSPAN22 (periphern2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, TSPAN34; and the anti-TLR antibody, or functional fragment thereof, specifically binds a toll-like receptor selected from the group consisting of cell surface expressed TLRs (TLR2, TLR4, TLR5, TLR6) or endosomal TLRs (TLR3, TLR7, TLR8, TLR9). In some embodiments, the tetraspanin is TSPAN30 (CD63).

In some embodiments, the anti-TLR antibody, or functional fragment thereof, inhibits the binding of an endosomal TLR to a ligand of the TLR. In some embodiments, the anti-TLR antibody, or functional fragment thereof, inhibits dimerization of an endosomal TLR. In some embodiments, the anti-TLR antibody, or functional fragment thereof, inhibits endosomal TLR activation via competitive or non-competitive Inhibition.

In some embodiments, the fusion protein comprises a peptidyl linker comprising a cleavage site of protease/endopeptidase. In some embodiments, the cleavage site is recognized by a protease/endopeptidase selected from the group consisting of cathepsin B, cathepsin H, cathepsin K, cathepsin L, cathepsin S, asparagine endopeptidase, and furin-like protease. In some embodiments, the peptidyl linker comprises a Z-Loop.

Some embodiments disclosed herein provide a pharmaceutical composition comprising the engineered extracellular vesicle described in the immediately preceding paragraph, and a pharmaceutically acceptable carrier or excipient. Some embodiments disclosed herein provide a method of treating an inflammatory response in an individual in need thereof, the method comprising administering to the individual therapeutically effective amount of a pharmaceutical composition an engineered extracellular vesicle described in the immediately preceding paragraph, and optionally an acceptable carrier or excipient.

Also provided herein are methods of treating, ameliorating, reducing, inhibiting an inflammatory response in an Individual in need thereof (such as because of viral Infection, and the like), the method comprising administering to the individual a therapeutically effective amount of a pharmaceutical composition comprising an engineered exosome described herein.

In some embodiments, the inflammatory response is a symptom of viral infection, bacterial Infection, autoimmune disease or chronic immune activation. In some embodiments, the composition is suitable for the treatment of an inflammatory response as a symptom of one or more disease or malady selected from the group of: COVID-19, cytokine storm, Systemic Lupus Erythematous (SLE), psoriasis, eczema, seborrheic dermatitis, actinic keratosis, glomerulonephritis, Sjögren's syndrome, systemic inflammatory syndrome (SIRS, e.g. sepsis with and without documented pathogen), macrophage activation syndrome (MAS), severe acute respiratory syndrome (SARS), hantavirus pulmonary syndrome, disseminated vascular coagulopathy (DIC), glomerulonephritis, diabetes (Type 1 and 2), traumatic brain injury, transplant, graft vs. host disease, liver fibrosis, pulmonary fibrosis, chronic obstructive pulmonary disease, asthma, influenza, human immunodeficiency virus (HIV) infection, hepatitis infection, viral pneumonia, rheumatoid arthritis, acute lung injury, crush injury, traumatic injury, bone fracture, metabolic syndrome, atherosclerosis, Addison's Disease, pheochromocytoma, metastasis, hyperacute insect sting, anaphylaxis, necrosis associated with flesh-eating bacterial Infection, kidney fibrosis, lupus nephritis, radiation therapy, frostbite, ischemia, reperfusion, myocardial infarction, bacterial pneumonia, bacterial sepsis, Legionnaire's disease and myocarditis and the like. In some embodiments, the inflammatory response is mediated by endosomal TLRs or cell surface expressed TLRs.

In some embodiments, the inflammatory response is mediated by activation of TLRs. In other embodiments, the methods described herein are useful for treating, ameliorating, reducing, inhibiting a proinflammatory response, which is MyD88-dependent (TLR2, TLR4, TLR5, TLR6, TLR7, TLR8, TLR9) and TRIF-dependent (TLR3). It is also contemplated herein that the described methods of treatment are useful when the activation of endosomal TLRs leads to increased activity of MAP kinases (ERK1/2, JNK, p38), IRAK, TRAF6, nuclear translocation of NF B. and increased activities of proinflammatory cytokines.

The described methods are also useful for the reduction of proinflammatory cytokines (TNF<, IL-1, IL-6, IL-12) and/or rise in anti-inflammatory cytokines (IL-10, IL-4, TGF-@. In addition, the methods described herein are also contemplated to reduce the level of interferons, such as type 1 and type 3, and the like.

Some embodiments described herein provide a method of treating, preventing or delaying the onset of cytokine storm in an Individual suspected or at risk of developing SIRS, MAS, or DIC, the method comprising administering to the individual, preferably prophylactically, an effective amount of an engineered exosome or a pharmaceutical composition described herein. In certain embodiments, severe infection-elicited cytokine storm is inhibited, thereby avoiding sepsis.

Some embodiments described herein provide a method of treating, preventing or delaying the onset of cytokine storm in an individual suspected or at risk of developing septic shock, the method comprising administering to the individual a prophylactically effective amount of an exosome or a pharmaceutical composition as described herein.

Additionally, the present invention may feature a method of preventing the production of anti-drug antibodies, in an Individual treated with biologics or a method of preventing immunosuppression in an individual treated with anti-TNFα biologic.

Some embodiments described herein provide an exosome-based delivery system for delivering a therapeutic molecule to a cell, wherein said delivery system comprises a fusion protein comprising a therapeutic molecule comprising an ant-TLR antibody, or functional fragment thereof, fused with a tetraspanin, or functional fragment thereof, wherein said tetraspanin, or functional fragment thereof, is positioned at a surface of an exosome, and wherein said tetraspanin, or functional fragment thereof, is capable of binding to a target on a recipient cell, thereby delivering the therapeutic molecule to the cell.

Also provided herein, is an exosome-based delivery system for delivering a therapeutic molecule (such as an anti-TLR antibody, and the like) fused with a tetraspanin (e.g., CD63, and the like) at the surface of exosome for binding to a target on recipient cell. In some embodiments, the targeting ligand is a fusion protein, wherein the fusion protein comprises a tetraspanin family protein and a moiety expressed in one of the extracellular loops (e.g. EC2 domain, NH2 domain) of the tetraspanin family protein.

In some embodiments, the tetraspanin member of the tetraspanin family of proteins is selected from the group consisting of: TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 1B), TSPAN21 (uroplakin1A) TSPAN22 (peripherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, and TSPAN34.

Some embodiments described herein provide a fusion protein comprising a tetraspanin, or a functional fragment thereof, linked to an anti-TLR antibody, or a functional fragment thereof, that inhibits cell surface expressed TLRs or endosomal TLR.

In some embodiments, the tetraspanin is selected from the group consisting of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 1B), TSPAN21 (uroplakin1A) TSPAN22 (peripherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, TSPAN34; and the anti-TLR antibody, or functional fragment thereof, specifically binds a toll-like receptor, or functional fragment thereof, wherein the toll-like receptor is selected from the group consisting of TLR2, TLR3, TLR4, TLR5, TLR6, TLR7, TLR8, and TLR9. In some embodiments, the tetraspanin, or functional fragment thereof is TSPAN30 (CD63), or a functional fragment thereof.

In some embodiments, the anti-TLR antibody, or functional fragment thereof, inhibits the binding of an endosomal or cell surface expressed TLR to a ligand of the TLR. In some embodiments, the anti-TLR antibody, or functional fragment thereof, inhibits dimerization of an endosomal TLR.

In some embodiments, the fusion protein further comprises a peptidyl linker comprising a cleavage site of protease/endopeptidase. In some embodiments, the cleavage site is recognized by a protease/endopeptidase selected from the group consisting of cathepsin B, cathepsin H, cathepsin K, cathepsin L, cathepsin S, asparagine endopeptidase, and furin-like protease. In some embodiments, the peptidyl linker comprises a Z-loop. In some embodiments, the fusion protein is selected from the group of fusion proteins set forth in Table 3 that have an anti-TLR antibody, or a functional fragment thereof, therein.

Some embodiments described herein provide a method for delivering a therapeutic molecule to an individual in need thereof. In some embodiments, the method comprises harvesting exosomes from a cultured packaging cell line, wherein the packaging cell line comprises the expression vector encoding a fusion protein as described herein. In some embodiments, the method further comprises administering the engineered exosome harvested previously to an individual in need thereof.

Some embodiments described herein provide a fusion protein comprising a tetraspanin, or a functional fragment thereof, linked to a functional moiety, wherein said fusion protein is selected from the group of fusion proteins set forth in Table 3.

Some embodiments described herein provide a dominant-negative engineered extracellular vesicle, wherein said extracellular vesicle comprises: a tetraspanin, or functional fragment thereof; a cleavable linker; and a dominant-negative fragment of a TLR receptor.

In some embodiments, the tetraspanin, or functional fragment thereof, is selected from the group consisting of: TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 1B), TSPAN21 (uropakin1A) TSPAN22 (peripherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, and TSPAN34, or a functional fragment of any one of the foregoing. In some embodiments, the dominant-negative fragment of a TLR receptor is a dominant-negative fragment of a toll-like receptor selected from the group consisting of TLR2, TLR3, TLR4, TLR5, TLR6, TLR7, TLR8, and TLR9.

Provided herein are engineered exosomes with an anti-TLR3, TLR7, TLR8 or TLR9 payload delivered into the endosome via tetraspanin-mediated endocytosis. This antibody: 1) inhibits the binding of endosomal TLRs to their respective ligands; 2) inhibits the dimerization of endosomal TLRs after ligand recognition; and/or 3) competitively or non-competitively inhibits TLR-ligand interaction.

In certain embodiments, the antibody is fused to CD9, CD63, CD81, CD153 with a linker that is susceptible to cleavage to proteases/endopeptidases activated in an acidic environment in endosome. Such proteases/endopeptidases include but are not limited to: cathepsins B, H, K. L, or S; or endopeptidases, e.g. asparagine endopeptidase

### Example

The following is a non-limiting example of the present invention, it is to be understood that said example is not Intended to limit the present invention in any way. Equivalents or substitutes are within the scope of the present invention.

### Example 1

Genetically modify human cells to produce engineered exosomes that deliver antibodies into endosomes inhibiting endosomal TLR-mediated inflammation.

### Example 1.1 Exosome-Based Payload Encapsulation and Delivery

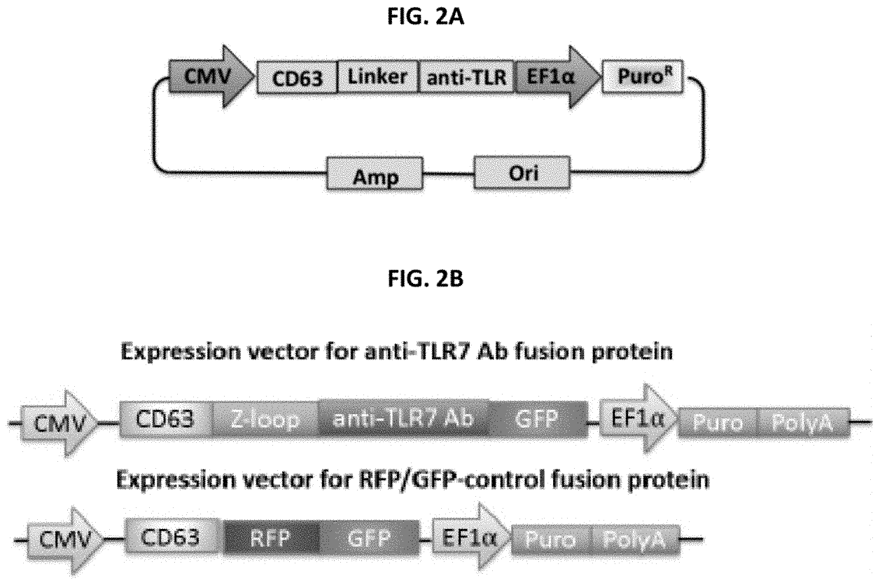

Tetraspanins are scaffold proteins expressed abundantly on the exosome surface. They are relatively small (220-350aa) and are used herein for exosomes display via molecular engineering in mammalian cells. In accordance with the present invention, an Innovative system is provided herein that delivers biologics that have been encapsulated inside engineered exosomes. Fusion protein constructs comprising fluorescent reporters or anti-endosomal-TLRs antibodies fused to the inner surface region of a tetraspanin, such as CD63, are constructed (FIGS. 2A-2B). The system according to the invention delivers large molecule therapeutics to intracellular targets that were previously undruggable by antibody biopharmaceuticals.

TLR3, 7, 8, and 9 reside in the endosomal membrane and, mediated by the ER membrane protein Unc93B1, the TLRs are transported from the endoplasmic reticulum to endolysosomes. In accordance with the present Invention, the engineered exosomes are effectively delivered to the endosomes. CD63 is a preferred targeting molecule because it is the most abundant surface marker of exosomes. Importantly, CD63 contains an intrinsic membrane localization signal, and traffics exosomes to the endocytic compartments. Using fluorescent reporters, the incorporation of the CD63 scaffold correctly onto exosomes and their delivery into endocytic compartments is monitored.

Next, TLRs 3, 7, 8and 9 possess along, inserted loop region (Z-loop) consisting of 45-60 amino acid residues (14 aa for TLR3; Table 1). Importantly, proteolytic cleavage at the Z-oop is believed to be indispensable for the function of TLRs, and cleavage site(s) lie within disordered regions of Z-loops in crystal structures. The methods and constructs described herein advantageously employ Z-loop as the cleavage site to release the payload (e.g., an scFv anti-TLR) after reaching the endosomal compartments. To that end, Z-loops are used in constructs as a cleavable linker between CD63 and payload (anti-TLR scFv) to exploit cleavage by endosomal proteases such as cathepsins, asparagine endopeptidases (AEP), furin-like proteases, and the like. The antibody-based payload is then untethered from the membrane, which facilitates anti-TLR payload binding to TLR receptor thus blocking inflammation induced by TLR-mediated signaling in the endosomes.

TABLE 1
Protease recognition site (bold) and known cleavage sites (arrows)
in Z-loops of TLRs.

| | | | Disordered | SEQ |
|---|---|---|---|---|
| TLR | Z-loop Sequence | Proteases | in Z-loop | ID NO: |
| TLR7 | NKISPSGDSSEVGFCSNARTSVESYEP | Cathepsins | PDB: 5GMF | 180 |
| | QVLEQLHYFRYDKYARSCRFKNKEAS | PMID: 21402738 | PMID: 27742543 | |
| | FMSVNES | | | |
| TLR8 | NRISPLVKDTRQSYAQSSSFQRHIRKR | PMID: 26929371 | PDB: 4R07 | 181 |
| | R↓STDFEFDPHSNFYHFTRPLIKPQ | | PMID: 25599397 | |
| TLR9 | NRISGASELTATMGEADGGEKVWLQP | Cathepsins, AEP | PDB: 3WPC | 182 |
| | GDLAPAPVDTPSSEDFRPN | (PMID: 21402738, | PMID: 25686612 | |
| | | 19879164) | | |
| Horse | NRISGAVEPVATTGEVDGGKKVWLTS | | | 183 |
| | RDLTPGPLDTPSSEDFMPSCKNLSF | | | |
| TLR3 | SFTKQSISLA↓SLPK | PMID: 25305318 | PD: 1ZIW | 184 |
| | | | PMID: 15961631 | |

### Example 1.2 Screening of Endosomal Anti-TLR Antibodies for Inhibition of Signaling by Intracellular Receptors

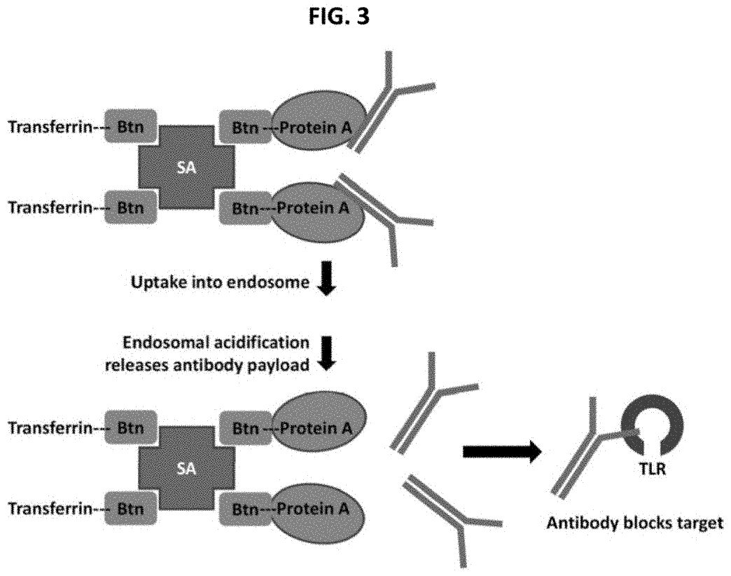

Protein complexes comprising (1) endosomally-trafficked transferrin, (2)streptavidin, (3) biotin-Protein A and (4) a series of anti-TLR antibodies, or TLR-binding fragments thereof (payload) are formed in vitro (FIG. 3). Screening assays using anti-TLR antibodies in protein complexes are used to measure inhibition of pro-inflammatory signaling by TLRs. In the signaling assay, reporter cell lines (including HEK TLR9 NFkB SEAP, HEK TLR8 NFkB SEAP, HEK TLR3 NFkB SEAP and control HEK NFkB SEAP) are pre-treated with protein complexes to promote uptake and trafficking into the endosome where the anti-TLR antibody payload is released to bind endosomal target (TLR receptor). Stimulation with the requisite TLR ligand results in phosphorylation of l1cB dissociating l1cB from NF1cB/l1cB complex resulting in NFkB translocating to the nucleus to induce expression of alkaline phosphatase (SEAP), which is secreted into the culture medium. Samples of culture medium are tested for SEAP enzymatic activity in alkaline buffer containing the fluorogenic substrate 4-methylumbelliferyl phosphate (FIG. 3). The first round of analysis utilizes protein complexes armed with polyclonal antibodies (against TLR9, TLR8, TLR7 or TLR3) to validate the approach for antagonizing each of these pathways.

### Example 1.2.1 Protein Complexes Bearing Anti-TLR9 Polyclonal Antibody Inhibit TLR9 Signaling in Reporter Cells

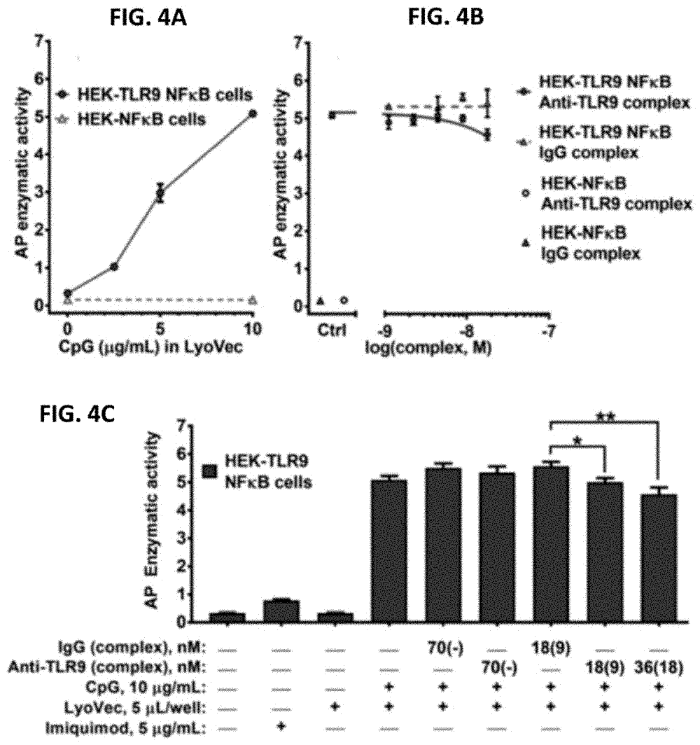

FIG. 4A shows that HEK-TLR9 NFkB cells that were treated with CpG DNA oligo (ODN 2006) in LyoVec, substantially as described above in Example 1.2, secreted SEAP into the medium (as shown by the initial rate of 4-MUP hydrolysis by AP enzyme), whereas HEK-NFkB control cells did not. FIG. 4B shows that HEK-TLR9 NFkB cells treated with CpG (10 μg/mL in LyoVec) secreted SEAP into the medium, but pre-treatment with protein complexes (containing endosome-trafficked transferrin) that bear the anti-TLR9 polyclonal antibody payload inhibited SEAP secretion in a concentration-dependent manner. Protein complexes bearing non-specific IgG had no effect. FIG. 4C, shows that neither the TLR7 agonist imiquimod nor LyoVec could stimulated TLR9 signaling in HEK-TLR9 NFkB cells. Pre-treatment with free antibody (either non-specific IgG or anti-TLR9 antibody) did not inhibit CpG/LyoVec-stimulated TLR9 signaling. Pre-treatment with the protein complex armed with control IgG did not inhibit TLR9 signaling with CpG/LyoVec, but by comparison, protein complexes loaded with anti-TLR9 polyclonal antibody payload demonstrated inhibition of signaling in a concentration dependent manner with 9 nM and 18 nM anti-TLR9 complex (*, p<0.05; * p<0.01).

### Example 1.2.2 Second Round Screening of anti-TLR Monoclonal Antibody Complexes

For TLR receptors that are inhibited, such as demonstrated for TLR9 above in Example 1.2.1, a second round is used to screen complexes armed with a series of monoclonal antibodies against the TLR (Table 2) and custom antibodies obtained from AvantGen (San Diego, Calif.). Hits identified from screening in cell assays (monoclonal antibodies that inhibit signaling) are tested for in vitro binding (pH 7 initially, later at endosomal pH 5.5) to TLRs using cell lysates (as TLR source) in ELISA kits [Human TLR3 ELISA Kit (Biogems cat. no. BGK6YOF1), Human TLR8 ELISA Kit (Aviva Systems Biology cat. no. OKEH01387), Human TLR8 ELISA Kit (MyBioSource cat no. MBS744532; LifeSpan Biosciences cat no. LS-F5812; Gentaur cat. no. 201-12-0376) or Human TLR9 ELISA Kit (MyBioSource cat. no. MBS751526; Antibodies Online cat. no. ABIN414673; Elabscience cat. no. E-EL-H0952) in which the secondary antibody-HRP conjugate is replaced with HRP (conjugated to Protein A. Protein G, anti-IgG as appropriate). High affinity binders at acidic pH are the Hit antibodies.

TABLE 2

| TLRs | Clone | Vendor |
|---|---|---|
| TLR3 | TLR3.7 | Thermo-Fisher |
| | 40C1285.6 | Invitrogen |
| TLR7 | A94B10 | BD Biosciences |
| | 4G6 | Invitrogen |
| TLR8 | 44C143 | Invitrogen |
| | 44B05 | Thermo-Fisher |
| TLR9 | 26C593.2 | Abcam |

Next, hits (anti-TLR antibody clones) are selected for fusion of the anti-TLR payload in the form of single chain variable fragment (scFv). DNA from positive clones in the AvantGen library is sequenced or protein sequence is obtained by proteolysis/Mass Spectrometry analysis using nSMOL Antibody BA Kit with MS analysis (both by Shimadzu) or by REmAb Antibody Sequencing service (Rapid Novor Kitchener, Ontario, CA). Lead antibodies are engineered as the payload (in the form of single-chain Fv (scFv)) as fusion proteins with tetraspanins (e.g., with an intervening cathepsin cleavage site) to generate prototype engineered exosomes for the drug delivery system described herein. Cleavage is optimized by testing a series of unlabeled peptides (containing cathepsin cleavage sites) in the presence of purified human cathepsin H and B (endosome-resident) in the presence of fluorogenic competitor substrate (Z-Arg-Arg-AMC, Sigma or similar (PMID 12201820)) to measure kinetic parameters (Km and kcat) of the unlabeled peptides [Case, 2003 #1453). Peptides with high selectivity (kcat/Km) for cathepsins H and/or B are engineered as the cathepsin site between tetraspanin and scFv payload. This novel payload encapsulation system provides an advantageous platform for supporting drug delivery and exosome-based therapy for delivery to intracellular targets that previously could not be targeted by biopharmaceuticals.

### Example 1.3 Stable Transfection and Production of Engineered Exosomes

The biological effects mediated by exosomal uptake in recipient cells depend on the donor cells. Exosomes produced by mature DCs carry major histocompatibility complex class ii (MHCII), intercellular adhesion molecule-1(ICAM-1) and co-stimulatory molecules and are potent T-cell stimulators. Mesenchymal stem cells (MSC) were clinically tested in humans and were immunosuppressive. MSCs are also the most prolific exosome producers; and are well suited for mass production of exosomes that are ideal for drug delivery in cases of inflammatory-driven autoimmune diseases.

MSC (ATCC cat. no. PCS-500-012) is stably transfected with a recombinant construct (FIGS. 2A-2B) to generate a stable cell line to produce exosomes. Engineered exosomes are generated from these stably transfected cells for further characterization. In other embodiments, a lab-scale hollow fiber bioreactor with protein-free culture medium can be used to obtain conditioned cell culture supernatant as a source of exosomes. Hollow fiber culture systems can sustain large numbers (>10′) of cells and produce highly concentrated cell culture supernatants in the manufacturing scale.

Using the encapsulation technology according to the invention to link a tetraspanin, such as CD63 as a molecular scaffold, to anti-TLR antibodies to encapsulate the payload inside exosomes results in preserving the natural targeting of the desired cells by exosomes. The novel technology provides a means for delivering anti-inflammatory molecules to specifically targeted pro-inflammatory cells rather than eliciting non-selective off-target effects. Since the payload Is encapsulated inside exosomes, it Is hidden from recognition by the immune system thereby avoiding time dependent increases in autoantibody expression, which is a drawback with using current anti-TNF(biologicals. Furthermore, using the Z-loops as the linker-cleavage site, the anti-TLR antibody payload is released from its fusion to CD63 within the exosome allowing the free antibodies to bind the intracellular (endosomal) TLR target and block TLR signaling. The specificity of using engineered exosomes as a drug carrier in accordance with the present invention provides new and improved treatments of many inflammation-driven diseases, such as, e.g., psoriasis, rheumatoid arthritis, systemic lupus erythematous, and the like, without significant side effects on bystander cells or significant other off-target side-effects.

In other embodiments, the anti-TLR fused to a Fab region is employed. In other embodiments, cells can be treated with engineered exosomes bearing CD63 fusions to the extracellular domains of TLR3, 7, 8 or 9 (lacking the transmembrane and TIR domains) instead of scFv or Fab. After cleavage, the newly liberated soluble receptors undergo ligand-mediated 4 K heterodimerization with intact endosomal TLR and block pro-inflammatory signaling.

### Example 1A Production of Engineered Exosomes Expressing Tetraspanin Fusions to Dominant-Negative TLR Receptors

Exosomes are engineered with fusions to dominant-negative (DN) TLRs that are defective in signaling (instead of tetraspanin fused to scFv or Fab). Cells are treated with tetraspanin fused to DN TLRs which are trafficked to the endosome, where upon ligand engagement, the DN TLR protomer heterodimerizes with the endogenous TLR to form a defective receptor complex that cannot signal downstream. Cells are treated with engineered exosomes bearing, for example, (in order from N- to -terminus) CD63 (lacking the fourth transmembrane (0204-224) and third cytoplasmic domain (0225-238)) fused to a cleavable linker (Z-domain) fused to domains of TLR3 (lacking signal peptide and TRIF domain) or TLR7, 8, or 9 (lacking the signal peptide and TIR domains). Thus, as set forth in Table 4, the engineered exosomes for DN TLR3 express C063(0204-238)-Zkoop-TLR3(01-23, 0726-904) fusion; the engineered exosomes for DN TLR7 express CD63(0204-238)-Zloop-TLR7(01-26, 0861-1049) fusion; the engineered exosomes for DN TLR8 express CD63(0204-238)-Zloop-TLR8(01-26, 0849-1041) fusion; and the engineered exosomes for DN TLR9 would express CD63(0204-238)-Zloop-TLR9(01-25, 0840-1032) fusion. After proteolytic cleavage in the endosome, the N-terminus of TLR receptors is liberated, and the DN TLR monomer can undergo ligand-mediated heterodimerization with intact endosomal TLR and block signaling.

TABLE 4

| Dominant- | |
|---|---|
| Negative | |
| Toll-Like | |
| Receptor | |
| fusion | Fusion (N-terminus to C-terminus) |
| DN TLR3 | CD63(A204-238)-Zloop-TLR3(A1-23, A726-904) |
| DN TLR7 | CD63(A204-238)-Zloop-TLR7(A1-26, A861-1049) |
| DN TLR8 | CD63(A204-238)-Zloop-TLR8(A1-26, A849-1041) |
| DN TLR9 | CD63(A204-238)-Zloop-TLR9(A1-25, A840-1032) |

### Example 2 Biophysical and Functional Characterization of Engineered Exosomes

In accordance with the present invention, methods, systems and exosome compositions are provided to prevent inflammation-driven autoimmune diseases, such as, e.g., rheumatoid arthritis, psoriasis, SLE, and the like, caused by activation of endosomal TLRs. Engineered exosomes are delivered to the endocytic compartments with a therapeutic payload that neutralizes endosomal TLR-driven inflammation. Stable cells are engineered to harness the ability of the robust system of the present invention for continuous engineered exosome production, and the mechanism of uptake of the exosomes is evaluated.

### Example 2.1. Determine whether the biophysical characteristics of exosomes are affected by genetic modification

To determine whether the engineered exosomes mature and ultimately release into extracellular space, engineered exosomes are subjected to nanoparticle tracking analysis. The size distribution (hydrodynamic diameter), and particle concentration is estimated. Exosomes isolated from engineered cells tagged with fluorescent protein in the amino terminus of CD64 and exosomes isolated from unmodified control cells are subjected to nanoparticle tracking analysis for comparison, using NanoSight NS300 (Malvern Instruments, University of Arizona core facility). For transmission electron microscopy (TEM), engineered exosomes and control exosome samples are fixed with 2% glutaraldehyde (Sigma-Aldrich) and then adsorbed to formvar/carbon coated TEM copper grids (SPI, Cat #3420C-MB). Samples are then stained negatively with 1% Uranyl Acetate (Sigma-Aldrich) for 10 sec. The samples are evaluated on JEOL 1011 transmission microscope at 80 kV, and digital images are acquired using AMT camera system (University of Arizona, Core Facility). The next characterization is subjecting the samples to Western blotting and probe with anti-CD63 antibody. CD63 is selected because it is the most abundant surface marker in exosomes. These experiments are used to determine whether genetic modification affects the biophysical characteristics of engineered exosomes.

Results: Exosomes have reported sizes of 40-120 nm. Using nanoparticle-tracking analysis, the engineered exosomes display two major peaks between 40-120 nm that are consistent with the reported sizes of exosomes. In addition, minor peaks greater than 120 nm exist, suggesting heterogeneity of exosomes. Although the heterogeneous nature of exosomes is not new, heterogeneity is found to exist under certain conditions. There are no differences in exosome sizes between our engineered exosomes compared to exosomes isolated from unmodified cells. Electron microscopy of negatively charged stain engineered exosomes reveals cup-shaped membrane vesicles of 50-90 nm. No differences between our engineered exosomes and exosomes from unmodified cells are revealed by transmission electron microscopy. Western blotting of engineered exosomes probed with anti-CD63 shows a positive band, which is consistent with published reports. These experimental results show that modified exosomes exhibit similar biophysical characteristics as that of unmodified exosomes.

### Example 2.2: The Uptake of Engineered Exosomes is Characterized to Determine Whether they Inhibit Endosomal TLR-Driven Inflammation

Because many exosome proteins have been shown to interact with membrane receptors on target cells, it is contemplated herein that exosome uptake is dependent upon the signaling status of recipient cells and of the protein complement of the exosome. Internalization of exosomes occurs frequently at recipient cells via direct fusion with the plasma membrane or endocytosis. Exosomes are usually taken up into the endosomal compartments via endocytosis. It is contemplated herein that recipient cells, via endocytosis, take up the engineered exosomes and the normal function or trafficking of the tetraspanin protein is not affected. To test this hypothesis, the fluorescent protein GFP2 linked to tetraspanin CD63 is monitored. Exosome uptake into primary macrophages is monitored by adding engineered exosomes detectable by fluorescent microscopy. Acceptor monocyte-derived macrophages (MDMs) are plated and then combined with different doses of purified GFP2-tagged exosomes and incubated for different times (i.e. 1-2h). Exosomes from unmodified cells are used as control. Next, whether the uptake of engineered exosomes is energy-dependent process is determined.

Macrophages are added to labeled exosomes and Incubated at 40C and their capacity internalize exosomes is determined. Control cells are incubated at 37 C. Next, whether the uptake of exosomes by macrophages requires a functioning cytoskeleton is determined; macrophages treated with engineered exosomes are pre-treated with cytochalasin D (metabolite that depolymerize actin filament network). These experiments will show whether uptake of engineered exosomes is an energy-dependent process that requires functioning cytoskeleton, both of which are indicative of endocytic pathways.

Next, whether macrophage uptake of engineered exosomes is dependent on clathrin-mediated endocytosis is determined. Macrophages are pre-treated with or without chlorpromazine (prevents the formation of clathrin coated pits at the plasma membrane). These experiments demonstrate that clathrin-mediated endocytosis plays a role in engineered exosome uptake.

Whether engineered exosomes can deliver fusion proteins to the endosome and release payload via the cleavable linker is determined. MDM cells are treated with engineered exosomes bearing the EYFP-CD63-Zloop-GFP2 fusion (fusion controls: EYFP-CD63-GFP2, EYFP-CD63 and CD63-GFP2) and are imaged by confocal microscopy or fluorescence microscope or flow cytometry. Cells containing uncleaved fusion exhibit a FRET signal. To the extent that the fusion protein is cleaved as intended in the Z-loop by endosomal protease(s), the FRET signal is decreased, whereas direct excitation of EYFP produces an unchanged yellow emission (indicating intact EYFP), and direct excitation of GFP2 yields increased green emission (indicating after proteolytic cleavage that intact free GFP2 is liberated and diffused away from membrane-bound EYFP). Constructs tested in cleavage assays include Z-oops from TLR3, TLR7, TLR8 or TLR9 as cleavable linkers. Imaging after LysoTracker Blue dye treatment Indicates trafficking to acidic endosomal compartments without interfering with detection of EYFP, GFP2 or FRET emissions.

Next, whether engineered exosomes modulate the inflammatory responses driven by endosomal TLR activated macrophages is determined, which includes measuring TNF(, IL-1, IL-4, IL-6, IL-10, TGF®, IL-12 levels in cell-free supernatant and comparing macrophages treated with and without different poly(IC)-TLR3 (InvivoGen), ssRNA40-TLR7/8 (InvivoGen), and CpG ODN-TLR9 (Sigma) followed by addition of engineered exosomes. Cytokines are measured using Multiplex Cytokine Assay (Millipore). Unmodified exosomes are used as negative control. These experiments indicate that engineered exosomes inhibit endosomal TLR ligand-mediated cytokine release.

These experiments demonstrate that when purified GFP2-tagged exosomes are incubated with adherent macrophages, engineered exosomes are taken up and delivered into the endosomal compartments. After incubation of macrophages with engineered exosomes, macrophages become fluorescent with time; and increasing amount of exosome uptake leads to brighter fluorescence, indicating specific uptake. Exosome uptake is confirmed by flow cytometry by a showing that a mean fluorescence index (MFI) is proportional to the amount of GFP2-tagged exosomes added. In contrast, control macrophages without GFP2-displayed exosomes show no GFP2 fluorescent background. Using confocal imaging, macrophages that internalized fluorescent exosomes display punctate intracellular fluorescence pattern, consistent with compartmentalized endosomal endocytosis. Furthermore, exosomal uptake is an energy-dependent process that requires cytoskeletal rearrangement, both indicative of endocytic pathways. These experiments indicate that surface displayed exosomes are functional and can be taken up by macrophages without altering the behavior of the exosomes during uptake.

It is contemplated herein that endosomal TLR ligands induce a robust inflammatory response indicated by increased levels of inflammatory cytokines such as TNF(, IL-6, IL-12 but not IL-4, IL-10, TGF®. However, in the presence of engineered exosomes according to the present invention, induction of inflammatory cytokines TNF(, IL-6, IL-12 is severely Inhibited but not the anti-inflammatory cytokines IL-4, IL-10. TGF®. These experiments demonstrate that the payload encapsulation system of the present invention provides a novel platform for supporting drug delivery and exosome-based therapy for inhibiting endosomal-driven inflammatory responses, and for treating the diseases caused by the endosomal-driven inflammatory responses

Table 3, which provides a Listing of Sequences is appended hereto.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An engineered extracellular vesicle comprising a fusion protein having tetraspanin, or a functional fragment thereof, linked to an anti-TLR antibody, or a functional fragment thereof, that serve as a targeting moiety and inhibits the activation of a cell surface expressed TLR to induce proinflammatory responses;
wherein the anti-TLR antibody is expressed on the exterior of the engineered extracellular vesicle.

2. The engineered extracellular vesicle of claim 1, wherein the tetraspanin is selected from the group consisting of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 18), TSPAN21 (uroplakin1A) TSPAN22 (peripherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, TSPAN34; and
the anti-TLR antibody, or functional fragment thereof, specifically binds a toll-like receptor selected from the group consisting of TLR1, TLR2, TLR4, TLR5, TLR6, and TLR10.

3. The engineered extracellular vesicle of claim 1, wherein the tetraspanin is TSPAN30 (CD63).

4. The engineered extracellular vesicle of claim 1, wherein the anti-TLR antibody, or functional fragment thereof, Inhibits the binding of a cell surface TLR to a ligand of the TLR.

5. The engineered extracellular vesicle of claim 1, wherein the anti-TLR antibody, or functional fragment thereof, Inhibits dimerization of a cell surface TLR.

6. The engineered extracellular vesicle of claim 1, wherein the anti-TLR antibody, or functional fragment thereof, targets and inhibits cell surface TLR activation via neutralization (competitive or non-competitive inhibition).

7. The engineered extracellular vesicle of claim 1, wherein the extracellular vesicle is an exosome.

8. A pharmaceutical composition comprising the engineered extracellular vesicle, comprising a fusion protein having tetraspanin, or a functional fragment thereof, linked to an anti-TLR antibody, or a functional fragment thereof, that inhibits a cell surface expressed TR, and a pharmaceutically acceptable carrier or excipient.

9. A method of treating an inflammatory response in an individual in need thereof, the method comprising administering to the individual therapeutically effective amount of a pharmaceutical composition of claim 8.

10. The method of claim 9, wherein the inflammatory response is a symptom of viral infections including COVID-19, MERS, SARS, HIV, gram positive and negative bacterial infections or chronic immune activation.

11. The method of claim 9, wherein the composition is suitable for the treatment of a disease or malady selected from the group of: cytokine storm, COVID-19. Sjögren's syndrome, systemic inflammatory syndrome (SIRS, e.g. sepsis with and without documented pathogen), macrophage activation syndrome (MAS), severe acute respiratory syndrome (SARS), hantavirus pulmonary syndrome, disseminated vascular coagulopathy (DIC), glomerulonephritis, diabetes (Type 1 and 2), traumatic brain injury, transplant, graft vs. host disease, liver fibrosis, pulmonary fibrosis, chronic obstructive pulmonary disease, asthma, influenza, human immunodeficiency virus (HIV) infection, hepatitis infection, viral pneumonia, rheumatoid arthritis, acute lung injury, crush injury, traumatic injury, bone fracture, metabolic syndrome, atherosclerosis, Addison's Disease, pheochromocytoma, metastasis, hyperacute insect sting, anaphylaxis, necrosis associated with flesh-eating bacterial infection, kidney fibrosis, radiation therapy, frostbite, ischemia, reperfusion, myocardial infarction, myocarditis, bacterial pneumonia, bacterial sepsis, and Legionnaires disease.

12. The method of claim 9, wherein the inflammatory response is mediated by cell surface TLRs activation.

13. A method of treating, preventing or delaying the onset of cytokine storm in an individual suspected or at risk of developing septic shock, the method comprising administering to the individual a prophylactically effective amount of pharmaceutical composition of claim 8.

14. An exosome-based delivery system for delivering a therapeutic molecule to a cell, wherein said delivery system comprises a fusion protein comprising a therapeutic molecule comprising an anti-TLR antibody, or functional fragment thereof, fused with a tetraspanin, or functional fragment thereof, wherein said tetraspanin, or functional fragment thereof, is positioned at a surface of an exosome, and wherein said anti-TLR antibody, or functional fragment thereof, will serve as a targeting moiety and neutralizing its effects by inhibiting dimerization of TLR receptors thereby dampening proinflammatory responses.

15. The exosome-based delivery system of claim 14, wherein the tetraspanin member of the tetraspanin family of proteins is selected from the group consisting of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 18), TSPAN21 (uroplakin1A) TSPAN22 (perpherin2), TSPAN23 (retinal outer segment membrane protein1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31, TSPAN32, TSPAN33, and TSPAN34.

16. A fusion protein comprising a tetraspanin, or a functional fragment thereof, linked to an anti-TLR antibody, or a functional fragment thereof, that inhibits cell surface expressed TIR-mediated activation.

17. The fusion protein of claim 16, wherein the tetraspanin is selected from the group consisting of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20 (uroplakin 11), TSPAN21 (uropiakin1A) TSPAN22 (peripherin2), TSPAN23 (retinal outer segment membrane protein1), TSPAN24 (CD151), TSPAN25 (CD53), TSPAN28 (CD37), TSPAN27 (CD82), TSPAN28 (CD81), TSPAN29 (CD9), TSPAN30 (CD63), TSPAN31, TSPAN32, TSPAN33, TSPAN34; and the anti-TLR antibody, or functional fragment thereof, specifically binds a toll-like receptor, or functional fragment thereof, wherein the toll-like receptor is selected from the group consisting of TLR1, TLR2, TLR4, TLR5, TLR6, and TLR10.
